(19) United States
(12) Patent Application Publication
(10) Pub. No.: US 2026/0166081 A9
(43) Pub. Date: Jun. 18, 2026

(54) INNATE LYMPHOID CELLS FOR CELL THERAPY AND BIOMARKERS THEREFOR

(71) Applicant: UNIVERSITY HEALTH NETWORK, Toronto (CA)

(72) Inventors: Sarah CROME, Toronto (CA); Kyle REID, Toronto (CA); Jessica MATHEWS, Toronto (CA); Julia MURPHY, Toronto (CA); Sarah COLPITTS, Toronto (CA)

(21) Appl. No.: 18/877,114

(22) PCT Filed: Jun. 20, 2024
(86) PCT No.: PCT/CA2023/050856

§ 371 Date: Dec. 19, 2024

Related U.S. Application Data
(60) Provisional application No. 63/353,823, filed on Jun. 20, 2022.
(60) Provisional application No. 63/469,234, filed on May 26, 2023.

(51) Int. Cl. A61K 35/17; A61K 40/10; A61P 37/06; C12N 5/078

(52) U.S. Cl. CPC A61K 35/17; A61K 40/10; A61P 37/06; C12N 5/0634

(57) ABSTRACT

There is described herein methods of ameliorating, treating or preventing graft-versus-host disease, transplant rejection or an autoimmune disorder, or promoting transplant graft function in a human using innate lymphoid cells (ILCs), including compositions comprising ILCs and methods for making the same.

### RELATED APPLICATIONS

This application claims priority to U.S. Provisional Application Nos. 63/353,823 and 63/469,234, filed Jun. 20, 2022 and May 26, 2023 respectively, both of which are incorporated herein by reference in their entirety.

### FIELD OF THE INVENTION

The invention relates to innate lymphoid cells (ILCs), methods for making the same, and their use in cell therapy.

### BACKGROUND OF THE INVENTION

Development of Graft-Versus-Host Disease (GVHD) following hematopoietic stem cell transplant (HSCT) is a major impediment to the success of this therapy in treating hematological malignancies. Following HSCT, donor stem cells develop into lymphocytes and attack cancerous cells, termed the Graft-Versus-Leukemia (GVL) effect. While HSCT is often a curative treatment, up to 80% of patients develop acute GVHD (aGVHD), characterized by donor T cells attacking healthy recipient tissues¹. 30% of aGVHD patients do not respond to current treatments involving systemic steroid therapy, and less than one third of non-responders survive past one year². While cell-based therapies using polyclonal regulatory T cells (Tregs), mesenchymal stromal cells (MSCs) or myeloid derived suppressor cells (MDSCs) have been widely pursued, clinical trial results to date have been only modestly successful³⁻⁵. Together, this highlights the need for more effective therapies.

Innate lymphoid cells (ILCs) are a family of innate lymphocytes with key roles in tissue homeostasis and immunity⁶. While ILCs are primarily tissue resident, they are also found in all secondary lymphoid tissues, where they impact various stages of an immune response⁶. ILC family members are defined by expression of signature cytokines and transcription factors^{6,7}. NK cells produce IFN-γ and TNF-α, require expression of T-box transcription factor (T-BET) and Eomesodermin (EOMES), and have central roles in anti-viral and anti-tumor immunity⁶. In humans, NK cells are further differentiated as being CD56^{dim}CD16⁺ NK cells that express killer cell immunoglobulin-like receptors (KIRs) and have strong cytotoxic potential, and CD56^{bright}CD16⁻ NK cells which lack KIR expression but produce high levels of IFN-γ and TNF-α⁸. Non-cytotoxic helper ILCs have also been identified. Group 1 ILCs (ILC1s) produce IFN-γ in a T-BET-dependent manner but do not express EOMES and contribute to immunity towards intracellular bacteria and viruses. Group 2 ILCs (ILC2s) express GATA-3, produce IL-4, IL-5, IL-9 as well as IL-13, and have important roles in type 2 immunity towards extracellular parasites. Group 3 ILCs (ILC3s) require expression of RORC2 and produce IL-22 alone or together with IL-17 and GM-CSF and are part of cellular circuits that respond to extracellular bacteria. Lymphoid Tissue inducer (LTi) cells express ILC3-associated transcription factors and cytokines, but also express surface Lymphotoxin (sLT) and LTB and help form secondary lymphoid structures^{6,7}.

Several studies have linked different ILC family members to a reduction in the incidence of acute graft-versus-host disease (aGVHD) following allogeneic hematopoietic stem cell transplant (HSCT). Early Natural Killer (NK) cell reconstitution is associated with increased patient survival, particularly expansion of CD56^{bright} NK cells^{9,10}. The presence of activated ILC2 and ILC3s were associated with a reduction in aGVHD incidence after allogeneic HSCT¹¹. In mouse, gastrointestinal ILC3s improve epithelial cell growth via IL-22 stimulation of intestinal stem cells post-bone marrow transplantation and suppress T cell proliferation via CD39- and CD73-mediated adenosine production^{12,13}. Intriguingly, Bruce et al. showed that the adoptive transfer of mouse ILC2s after allogeneic bone marrow transplant improved survival of the mice and limited intestinal GVHD by recruiting myeloid derived suppressor cells (MDSCs), resulting in reduced inflammatory donor T cells and improved intestinal barrier function¹⁴.

In addition to GVHD, ILC2s have been reported to limit harmful immune responses across multiple organs, either through direct regulation of inflammatory cells or via recruitment of other immunosuppressive cells¹⁵. Recent studies identified an ILC2 subpopulation that produces IL-10 (ILC2₁₀) and directly regulates immune responses¹⁵. Seehus et al. demonstrated IL-2 induced IL-10 production by lung ILC2s that served as tissue sentinels capable of rapidly controlling lung inflammation in mice; IL-10 production in lung ILC2s driven by Blimp-1 and cMaf expression suppressed airway hyperreactivityl^{6,17}; Murine meningeal IL-10 producing ILC2s ameliorated neuroinflammation; and intestinal ILC2s can be induced to produce IL-10 through various tissue molecules^{18,19}. Clinically, ILC2₁₀ have been linked to positive responses to allergy immunotherapy in humans and can help sustain epithelial barrier integrity and suppress helper T cell responses²⁰. Collectively, these studies support that ILC2₁₀ in particular have important functions in regulating harmful immune responses.

### SUMMARY OF THE INVENTION

In some examples of the present study, we asked whether human ILC2s have immunoregulatory applications for GVHD and transplantation after observing an inverse relationship between ILC2s and CD4⁺ T helper 1 (Th1) or CD8⁺ cytotoxic T cells (Tc1) in HSCT recipients that corresponded with protection or susceptibility to GVHD. ILC2s expanded ex vivo using our approach expressed high levels of IL-10, in line with a regulatory ILC2₁₀ phenotype, and limited GVHD by suppressing allogeneic T cell responses. We identified mechanisms whereby IL-10-producing human ILC2s restrained allogeneic T cell responses. We asked whether human ILC2₁₀ have cell therapy applications using humanized mouse models of GVHD. We further examined whether ILC2s were associated with cancer relapse in HSCT recipients or would impair GVL responses. Collectively, these studies support IL-10-producing ILC2s may have applications in cell-based therapies aimed at dampening harmful allogeneic immune responses.

Accordingly, in an aspect, there is provided a method of ameliorating, treating or preventing graft-versus-host disease, transplant rejection or an autoimmune disorder, or promoting transplant graft function in a human subject in need thereof, the method comprising administering to the subject a therapeutically effective amount of a population of cells comprising innate lymphoid cells (ILCs).

In an aspect, there is provided a pharmaceutical composition comprising innate lymphoid cells (ILCs) for use in ameliorating, treating or preventing graft-versus-host disease or graft rejection, or an autoimmune disorder, or promoting transplant graft function, in a human subject in need thereof.

In an aspect, there is provided a use of innate lymphoid cells (ILCs) in the preparation of a medicament for ameliorating, treating or preventing graft-versus-host disease or graft rejection, or an autoimmune disorder, or promoting transplant graft function, in a human subject in need thereof.

In an aspect, there is provided a method for inducing a population of regulatory innate lymphoid cells, (ILCregs) from innate lymphoid cell (ILC) precursors and/or NK cells, and the method comprising culturing the population with at least one of TGF-β, IL-2 and anti-IFN-γ.

In an aspect, there is provided a population enriched for ILCregs produced by the methods described herein.

In an aspect, there is provided a method of selecting from a population of cells comprising innate lymphoid cells (ILCs), those cells that are ILC2 cells by selecting for cells that are CD45+, CD127+, CRTh2+ and CD3−, CD4−, CD8−, CD14−, CD15−, CD19−, CD20−, CD33−, CD34−, CD68−, CD138−, CD203c−, FCεRI−, TCRαβ−, TCRγδ−, IgA−, CD16−, CD94−, NKG2D−, CCR6− and NKp44−.

In an aspect, there is provided a method of selecting from a population of cells comprising innate lymphoid cells (ILCs), those cells that are ILC3 cells by selecting for cells that are CD45+, CD127+, CD117+, CCR6+, CRTh2−, CD3−, CD4−, CD8−, CD14−, CD15−, CD19−, CD20−, CD33−, CD34−, CD68−, CD138−, CD203c−, FCεRI−, TCRαβ−, TCRγδ−, IgA−, CD16−, CD94− and NKG2D.

In an aspect, there is provided a method for expanding innate lymphoid cells (ILCs), from a sample comprising ILCs, the method comprising: expanding isolated ILCs in an expansion medium supplemented with one or more of IL-2, IL-7, and IL-33, for ILC2s and/or IL-2, IL-7, IL-1B, and IL-23 for ILC3s.

### BRIEF DESCRIPTION OF FIGURES

These and other features of the preferred embodiments of the invention will become more apparent in the following detailed description in which reference is made to the appended drawings wherein:

FIG. 1: Total Helper ILCs and ILC2s Increased in Peripheral Blood in HSCT Patients that Do Not Develop acute GVHD. PBMCs from healthy donors, and HSCT patients with or without GVHD were assessed for ILC proportions and phenotypes using flow cytometry. Representative flow plots (a) and summary graphs (b) of CD56^{dim} CD16⁺ and CD56^{bright} CD16-NK cells in PBMCs, as a proportion of live CD45⁺ lineage negative cells. Lineage markers are CD3, CD4, CD8, CD14, CD19, CD20, CD33, CD34, CD123, CD138, CD303, FCεRI and TCRγδ. c, d, Representative flow plots (c) and summary graphs (d) of total CD127⁺ helper ILCs in PBMCs as a proportion of live CD45⁺ lineage negative cells. e,f, Representative flow plots (e) and summary graphs (f) of CD117⁻, CRTh2⁻, CXCR3⁺ ILC1s, CRTh2⁺ ILC2s and CRTh2⁻ CD117⁺ ILC3s in PBMCs as a proportion of live CD45⁺ lineage negative cells. (g) Representative flow plots of CD4⁺ Th1 and CD8⁺ Tc1 cells in HSCT patients with or without aGHVD development. (h) CD4⁺ Th1 and CD8⁺ Tc1 cells across HSCT patients as a proportion of CD4⁺ and CD8⁺ T cells respectively. (i) Correlation of Th1 and Tc1 cells with ILC2s in all HSCT patients.

FIG. 2: Human ILCs Isolated from Healthy Blood Rapidly Expand in vitro and Maintain Expression of Signature Cytokines. To explore the role of human ILC2s in GVHD, human ILCs had to be isolated from peripheral blood using flow cytometry and expanded ex vivo using ILC subset-specific cytokines as they are present in very low abundance in peripheral blood. (a) Overview of isolation and expansion of human ILC subsets. Briefly, total PBMCs are isolated from healthy donor blood. PBMCs are incubated with FITC-conjugated linage antibodies. Lineage antibodies added are CD3 (OKT3), CD3 (UCHT1), CD4, CD8a, CD14, CD15, CD19, CD20, TCRαβ, TCRγδ, CD33, CD34, CD203c, FCεRI, CD79a and CD138. Anti-FITC magnetic microbeads are added and cells are enriched for lineage cells using StemCell magnets. Enriched cells are FACS sorted for CD56^{dim} NK cells, ILC2s and ILC3s. ILC2s are cultured with 100U/mL IL-2, 10 ng/ml IL-7 and 10 ng/ml IL-33, ILC3s are cultured with 100U/ml IL-2, 10 ng/ml IL-7, 10 ng/ML IL-1β, and 10 ng/ml of IL-23 and CD56^{dim} NK cells are cultured with 500U/mL IL-2, 10 ng/ML IL-15, and 10 ng/ml of IL-18. (b) Representative gating strategy for ILC2s and ILC3s. ILC2s sorted as live, lineage⁻ CD94, NKG2D, CD127⁺ with ILC2s being CRTh2⁺ CCR6 and ILC3s being CRTh2⁻, CD117⁺, CCR6⁺. (c) Cell expansion counts of ILC2s and ILC3s over 20 and 33 days. Representative plots (d) and summary plots (e) of intracellular cytokine staining for signature cytokines in CD56^{dim} NK cells, ILC2s and ILC3s expanded for 20 days after stimulation with phorbol 12-myristate 13-acetate and ionomycin and blocked with monensin and brefeldin A. (f) Cytometric bead array analysis of secreted cytokines after plating at a concentration of 2×10⁵ cells/mL for 16-hour stimulation with 100U/ml of IL-2.

FIG. 3: Expanded Human ILC2s Suppress Symptoms and Improve Survival of Xenograft GVHD. To assess whether ILC2s may have the capacity to directly limit GVHD, we employed an established xenograft model of GVHD involving PBMC transfer into NOD-scid-IL2Rg^{null} mice. (a) Overview of the xenograft GVHD model. Briefly, NOD-scid-IL2Rg^{null} mice are irradiated and either injected with PBS, human PBMCs to induce multiorgan tissue pathology, or PBMCs with ILC2s. Mice are monitored for symptoms of xenograft GVHD including weight loss and a combined xenograft GVHD score as well as survival. Blood is drawn at 7-day intervals for immunophenotyping and engraftment. At the humane or experimental endpoint, mice are euthanized and organs are taken for histology. The blood, bone marrow and spleens along with the small and large intestines are taken for flow cytometry phenotyping. b,c, Representative plots (b) and summary graphs (c) showing engraftment of human CD3+CD45+ T cells at day 14 post injection of human PBMCs (n=11-13). d, Engraftment of human CD4+ and CD8+ human T cell subsets in the blood at day 14 post injection (n=11-13). e, Proportion of CD4+ and CD8+ T cells of total T cells in the blood, bone marrow and spleens of mice at humane endpoint (n=11-13). f, Representative xenograft GVHD score plots (n=3) from three independent experiments. g, Representative weight loss plots (n=3) from three independent experiments. h,i Summary dot plots of xenograft GVHD score (h) and weight loss (i) of mice at day 20 post injection (n=14-23).

FIG. 4: ILC2s Suppress Proliferation and Reduce CXCR3 Expression on CD4⁺ and CD8⁺ T cells in vivo. Effect of ILC2 transfer on T cell phenotypes and proliferation in xenograft GVHD model were assessed by flow cytometry. (a), Representative plots showing expression of Ki-67 on CD4⁺ T cells from the blood at end point. (b) Ki-67 expression in blood, bone marrow or spleen at endpoint on CD4⁺ T cells (n=11 from n=3 independent experiments). (c) Representative plots of Ki-67 expression by flow cytometry on CD8⁺ T cells from the blood. (d) Ki-67 expression in the blood, bone marrow or spleen at endpoint on CD8⁺ T cells (n=11 from n=3 independent experiments). (e) Representative human CD3 immunohistochemistry of the colon of mice treated with PBS, PBMCs or PBMCs+ILC2s. (f) Average T cell infiltration in the colon of mice treated with PBS, PBMCs or PBMCs+ILC2s. Quantified using CD3 immunohistochemistry and the HALO algorithm and normalized to background in PBS mice. g, h, Representative flow plots of CXCR3 expression on blood CD4⁺ (g) or CD8⁺ (h) T cells. CXCR3 expression on CD4⁺ (i) or CD8⁺ (j) T cells in the blood, bone marrow, spleen, colon, and small intestines at ethical endpoint (n=5-8).

FIG. 5: ILC2s Suppress CD4⁺ and CD8⁺ Inflammatory Cytokine Production in vitro. To confirm effects of ILC2 transfer was due to direct effects on ILC2s on allogeneic CD4⁺ and CD8⁺ T cells, we performed in vitro co-culture assays with expanded ILC2s and naïve CD4⁺ and CD8⁺ T cells. After 4 days in culture, intracellular cytokine staining was assessed by flow cytometry. (a) Representative IFN-γ and TNF-α expression on naïve CD4⁺ T cells co-cultured with allogeneic ILC2s and stimulated with anti-CD3/CD28 beads for 4 days. (b) Average decrease in IFN-g production or IFN-g and TNF-α co-expression represented as log 2fold change compared to CD4⁺ T cells alone (n=17). (c) Representative IFN-γ and TNF-α expression on CD8⁺ T cells co-cultured with allogeneic ILC2s and stimulated with anti-CD3/CD28 beads over 4 days by representative flow cytometry plots. (d) Average decrease in IFN-g and co-expression of IFN-g and TNF-α representative as log₂fold change compared to CD8⁺ T cells alone (n=8).

FIG. 6: Expanded ILC2s regulate CD4⁺ and CD8⁺ T cells via a combination of IL-4 and IL-10. (a) UMAP plots of expanded ILC subsets after CITE-seq. ILC subsets were isolated using FACS, expanded and then separately stained with an antibody cocktail. Each cell population was stained with a unique hashtag antibody to allow identification post sequencing. (b) Feature plots showing protein level expression of CD16, CD56 and CD117 and RNA level expression of PTGDR2, GATA3, IL17RB, GNLY, IL13, IL5 and IL-10 on expanded ILC subsets. c, Representative flow cytometry plots for IL-10 and amphiregulin expression on expanded CD56^{dim} NK cells, ILC2s and ILC3s after PMA/lonomycin stimulation. (d) Expression of intracellular IL-10 from expanded ILC subsets by flow cytometry (n=11-13) and secreted IL-10 by cytometric bead array (n=12-19). (e) Violin plots of single cell expression of protein level CD39 and CD73 and RNA expression of ENTPD1 and NT5E after expansion. Flow cytometry expression of CD39 and CD73 on expanded ILC subsets shown as representative flow plots (f) and summary graphs (g) (n=10-20). (h) Representative flow plots of CD4⁺ and CD8⁺ T cell co-cultures with ILC2s and anti-IL-4 and/or anti-IL-10 antibodies. Changes in fold IFN-γ expression after co-culture with ILC2s with the addition of IL-4 and/or IL-10 antibodies on CD4⁺ (i) or CD8⁺ (j) T cells. Correlation of IL-10 expression with amphiregulin (k), IL-4 (l), IL-9 (m) or IL-13 (n) (n=22, 15, 18 and 13 respectively) on expanded ILC2s with ILC2₁₀ phenotype.

FIG. 7: Cell therapy with ILC2₁₀ does not impede T cell-mediated graft-versus-leukemia effect. (a) Circulating ILC2s from HSCT patients as a proportion of lineage negative PBMCs. Patients were grouped based on whether they experienced cancer relapse (n=5) or not (n=13). Follow up periods at time of assessment ranged from 30 to 120 days. (b) Overview of humanized GVL model, whereby MV4-11 AML cells are transplanted into NSG mice. PBMCs are administered at D5 following MV4-11 transfer, and engrafted T cells reduced MV4-11 cell engraftment. (c) Representative MV4-11 cells in bone marrow of NSG mice treated with PBS, PBMCs or PBMCs with ILC2₁₀. (d) Average MV4-11 engraftment in NSG mice treated with PBS, PBMCs or PBMCs with ILC2₁₀ from two independent experiments (n=7/group) (e) Average xenogeneic GVHD score at day 19 from 2 independent experiments (n=7/group). Day 19 corresponded to 14 days following injection of PBMCs with or without ILC2₁₀.

FIG. 8. Assessing the impact of Immunocult medium in comparison to XVIVO-15 for ILC2s and ILC3s. (a) Method of testing the effects of different mediums on ILC2s and ILC3s. (b) ILC2 and (c) ILC3 cell count and fold expansion at day 33. (d) Representative FAGS plots of intracellular cytokine staining after 6 hour of PMA/ionomycin stimulation on day 33. (e) Summary of cytokine expression (n=3).

FIG. 9. Preliminary results of AIM-V and StemSpan medium on ILC2 and ILC3. (a) Cell counts and fold expansion at day 33 for ILC2 and (b) ILC3s. (c) Representative FAGS plots of intracellular cytokines after 6 hour PMA/ionomycin stimulation. (d) Summary of cytokine expression (n=1).

FIG. 10. ILCs suppress antigen-specific islet rejection and regulate harmful T cell subsets. (a) NSG mice were injected with 150 mg/kg STZ to kill endogenous β cells and transplanted with 750 HLA-A2+ IEQ under the kidney capsule with or without 1.0×106 ILC2s or ILC3s and monitored for blood glucose levels. After 30 days, mice were transfused with 1.5×106 HLA-A2-specific CD4 and CD8 CAR-T cells with or without 1.0×106 ILC2s or ILC3s. On day 24 post-T cell infusion, mice were fasted for 4 hours and challenged with 1.5 g of glucose/kg. (b-c) HLA-A2-specific CAR T cells or Naïve CD4⁺ T cells isolated using FACS sorting were plated with ILC2s or ILC3s (1:1 ratio) and stimulated with anti-CD3/CD28 Dyna beads for 2 or 4 days, respectively. Surface and intracellular markers were quantified by flow cytometry. Intracellular cytokine production was quantified by flow cytometry after a 6-hour stimulation with PMA/Ionocmycin. (d-e) In vitro co-culture of ILC2₁₀ with HLA-A2+ CAR T cells demonstrates ILC2₁₀ directly inhibit T cell proliferation (Ki67) and CD4+ and CD8+ T cell production of proinflammatory cytokines IFN-g and TNF-a.

### DETAILED DESCRIPTION

In the following description, numerous specific details are set forth to provide a thorough understanding of the invention. However, it is understood that the invention may be practiced without these specific details.

Identification of cells and molecules that limit harmful immune responses is critical for the development of tolerance-promoting therapeutic strategies. Innate lymphoid cells (ILCs) rapidly respond to microenvironmental signals and orchestrate immune responses to maintain homeostasis. Following hematopoietic stem cell transplant (HSCT), reduced proportions of CD56^{bright} Natural Killer (NK) cells as well as Group 2 ILCs (ILC2s) and Group 3 ILCs (ILC3s) are observed in patients who develop acute graft-versus-host disease (aGVHD), and HSCT recipients that receive ILC-rich grafts have a lower risk of developing aGVHD. Here, we report that the presence of ILC2s correlates with decreased proportions of CD4⁺ T helper 1 (Th1) cells in HSCT recipients and protection from aGVHD, suggesting ILC2s may antagonize harmful T cell responses in GVHD. To assess the potential of human ILC2s to limit T cell responses in aGVHD, we developed methods to isolate and expand human ILC2s from healthy donor blood in a manner that maintains expression of their signature cytokines, IL-4, IL-5, IL-9 and IL-13. Using a xenograft model of aGVHD, we show that adoptive transfer of expanded human ILC2s reduces the severity of xenograft GVHD symptoms, reduces weight loss and prolongs survival of NOD-scid IL2Rγ^{null} mice. ILC2s limited proliferation of allogeneic CD4⁺ and CD8⁺ T cells and reduce the proportion of CD4⁺ Th1 and CD8⁺ Tc1 cells in vivo, in line with observations in HSCT patients. Immunohistochemistry revealed that there was also reduced T cell infiltration with adoptive transfer of ILC2s. Single cell RNA sequencing of expanded ILC populations identified IL-10 as a key cytokine differentially expressed by expanded human ILC2s, in addition to canonical ILC2 cytokines. In vitro studies demonstrated direct regulation of allogeneic T cells by human IL-10-producing ILC2s is mediated by a combination of IL-4 and IL-10. Cell therapy with human ILC2₁₀ decreased the severity of xenogeneic GVHD and prolonged NOD-scid IL2Rγ^{null} (NSG) mice survival when given prophylactically or upon GVHD onset. Collectively our findings support that human IL-10-producing ILC2s limit harmful allogeneic T cell responses and may have applications in adoptive cell-based therapies for GVHD.

In an aspect therefore, there is provided a method of ameliorating, treating or preventing graft-versus-host disease, transplant rejection or an autoimmune disorder, or promoting transplant graft function in a human subject in need thereof, the method comprising administering to the subject a therapeutically effective amount of a population of cells comprising innate lymphoid cells (ILCs).

As used herein, “therapeutically effective amount” refers to an amount effective, at dosages and for a particular period of time necessary, to achieve the desired therapeutic result. A therapeutically effective amount of the pharmacological agent may vary according to factors such as the disease state, age, sex, and weight of the individual, and the ability of the pharmacological agent to elicit a desired response in the individual. A therapeutically effective amount is also one in which any toxic or detrimental effects of the pharmacological agent are outweighed by the therapeutically beneficial effects.

In some embodiments, the population comprises ILC2 cells, ILC3 cells or ILCregs cells or combinations thereof.

In some embodiments, the population comprises ILC2 cells that produce IL-4, IL-5, IL-9 or IL-13.

In some embodiments, the ILC2 cells produce IL-10. Preferably, the ILC2, cells additionally produce Amphiregulin.

In some embodiments, the ILC2 cells are CD45+, CD127+, CRTh2+ and CD3−, CD4−, CD8−, CD14−, CD15−, CD19−, CD20−, CD33−, CD34−, CD68−, CD138−, CD203c−, FCεRI−, TCRαβ−, TCRγδ−, IgA−, CD16−,CD94−, NKG2D−, CCR6− and NKp44−.

In some embodiments, the population comprises ILC3 cells that produce IL-22 alone or in combination with IL-17 or GM-CSF. Preferably, the ILC3 cells additionally produce IL-10 and Amphiregulin. In some embodiments, the ILC3 cells are CD45+, CD127+, CD117+, CCR6+, CRTh2−, CD3−, CD4−, CD8−, CD14−, CD15−, CD19−, CD20−, CD33−, CD34−, CD68−, CD138−, CD203c−, FCεRI−, TCRαβ−, TCRγδ−, IgA−, CD16−, CD94− and NKG2D−.

In some embodiments, the population comprises ILCregs that are CD7+, NKp44+, NKp46+, express Galectin-9 and HELIOS, do not express IL-10, and have low to no expression of IFN-γ and TNF-α.

In some embodiments, the ILCs are CD56+CD3−.

In some embodiments, the ILCregs are further at least one of CD56hi, CD16−, CD7+, CD94+, NKG2D+, KIR+, GITR, NKp44− ex vivo, NKp46+, IL-22+, do not express IL-10 and have low to no expression of IFN-γ and TNF-α. Preferably, the ILCregs are further all of CD56hi, CD16−, CD7+, CD94+, NKG2D+, KIR+, GITR, NKp44− ex vivo, NKp30+, NKp46+, IL-22+, do not express IL-10 and have low to no expression of IFN-γ and TNF-α

In some embodiments, the ILCs suppress T-cell function or propagation.

In some embodiments, the subject has undergone or will undergo a cell transplantation procedure. Preferably, the cell transplantation is stem cell transplantation, the latter preferably being hematopoietic stem cell transplantation.

In some embodiments, the subject has undergone or will undergo a bone marrow transplantation procedure.

In some embodiments, the subject has undergone or will undergo a solid organ transplantation procedure.

In an aspect, there is provided a pharmaceutical composition comprising innate lymphoid cells (ILCs) for use in ameliorating, treating or preventing graft-versus-host disease or graft rejection, or an autoimmune disorder, or promoting transplant graft function, in a human subject in need thereof.

In an aspect, there is provided a use of innate lymphoid cells (ILCs) in the preparation of a medicament for ameliorating, treating or preventing graft-versus-host disease or graft rejection, or an autoimmune disorder, or promoting transplant graft function, in a human subject in need thereof.

In an aspect, there is provided a method for inducing a population of regulatory innate lymphoid cells, (ILCregs) from innate lymphoid cell (ILC) precursors and/or NK cells, and the method comprising culturing the population with at least one of TGF-β, IL-2 and anti-IFN-γ.

In some embodiments, the method comprises culturing the population with all of TGF-β, IL-2 and anti-IFN-γ. In some embodiments, the ILCreg population is induced from ILC precursors and the population is additionally cultured with IL-23, IL-1b, and IL-7.

Preferably, the NK cells are CD56+, preferably CD56^{bright}.

In some embodiments, the induced ILCregs are CD7+, NKp44+, NKp46+, express Galectin-9, HELIOS, do not express IL-10, and have low to no expression of IFN-γ and TNF-α.

In some embodiments, the induced ILCregs are CD56+CD3−. Preferably, the induced ILCregs are further at least one of CD56+, CD7+, NKp44+, NKp46+, express Galectin-9 and HELIOS, do not express IL-10, and have low to no expression of IFN-γ and TNF-α. Further preferably, the induced ILCregs are further all of CD7+, NKp44+, NKp46+, express Galectin-9 and HELIOS, do not express IL-10, and have low to no expression of IFN-γ and TNF-α.

In some embodiments, the ILCregs suppress T-cell function or propagation.

In an aspect, there is provided a population enriched for ILCregs produced by the methods described herein.

In an aspect, there is provided a method of selecting from a population of population of cells comprising innate lymphoid cells (ILCs), those cells that are ILC2 cells by selecting for cells that are CD45+, CD127+, CRTh2+ and CD3−, CD4−, CD8−, CD14−, CD15−, CD19−, CD20−, CD33−, CD34−, CD68−, CD138−, CD203c−, FCεRI−, TCRαβ−, TCRγδ−, IgA−, CD16−,CD94−, NKG2D−, CCR6− and NKp44−.

In some embodiments, the method further comprises expanding the selected ILC2s in X-VIVO15 media supplemented with IL-7, IL-33 and IL-2. Preferably, the X-VIVO15 media is supplemented with about 20 ng/ml of IL-7, about 20 ng/ml IL-33 and about 100 IU/ml of IL-2.

In an aspect, there is provided a method of selecting from a population of population of cells comprising innate lymphoid cells (ILCs), those cells that are ILC3 cells by selecting for cells that are CD45⁺, CD127⁺, CD117⁺, CCR6⁺, CRTh2−, CD3−, CD4−, CD8−, CD14−, CD15−, CD19−, CD20−, CD33−, CD34−, CD68−, CD138−, CD203c−, FCεRI−, TCRαβ−, TCRγδ−, IgA−, CD16−, CD94− and NKG2D.

In some embodiments, the method further comprises expanding the selected ILC3 cells in X-VIVO15 media supplemented with IL-1β, IL-7, IL-23, and IL-2. Preferably, the X-VIVO15 media is supplemented with about 20 ng/ml each of IL-1β, IL-7 and IL-23, and about 100 IU/mL of IL-2.

In an aspect, there is provided a method for expanding innate lymphoid cells (ILCs), from a sample comprising ILCs, the method comprising: expanding isolated ILCs in an expansion medium supplemented with one or more of IL-2, IL-7, and IL-33, for ILC2s and/or IL-2, IL-7, IL-1β, and IL-23 for ILC3s.

In some embodiments, the expansion medium is supplemented with all of IL-2, IL-7, and IL-33, for ILC2s and/or IL-2, IL-7, IL-1β, and IL-23 for ILC3s. Preferably, the expansion medium is X-VIVO 15. In some embodiments, the ILCs are ILC2s. In other embodiments, the ILCs are ILC3s.

In some embodiments, the isolated ILCS are first obtained by selecting cells with ILC specific cell markers from the sample. Preferably, isolating the ILCs from the sample comprises first gating helper ILCs as Live, Lineage-CD94-NKG2D-CD127+.

In some embodiments, isolating the ILCs from the sample comprises selecting for cells that are one or more of CRTh2+, CCR6− and CD56− for ILC2s and CRTh2−CD117+ and CCR6+ for ILC3s.

In some embodiments, isolating the ILCs from the sample comprises selecting for cells that are all of CRTh2+, CCR6− and CD56− for ILC2s and CRTh2−CD117+ and CCR6+ for ILC3s.

In some embodiments, the sample is patient blood sample.

The advantages of the present invention are further illustrated by the following examples. The examples and their particular details set forth herein are presented for illustration only and should not be construed as a limitation on the claims of the present invention.

### EXAMPLES

### Methods and Materials

### Study Participants

Study protocols were approved by the Research Ethics Board of the University Health Network in accordance with the Helsinki Declaration (UHN REB 19-6351). All patients provided written, informed consent for collection of samples. Fresh peripheral blood was collected in EDTA blood collection tubes (BD Biosciences). Frozen PBMC samples were obtained from the Messner Allogeneic Transplant Program Biobank.

### Human PBMC Isolation

Fresh peripheral blood was obtained from healthy donors or through the Canadian Blood Services Blood4Research program, with each donor providing written, informed consent (UHN REB 17-6229, CBS Approved Study 2020-047). Blood was diluted 1:1 with FACS buffer (1×PBS (Gibco) with 2% FBS (ThermoFisherScientific)) and layered on top of Lymphoprep (StemCell Technologies) as per manufacturer instructions. After removal of the PBMC layer, cells were washed in FACS buffer, platelets were removed by centrifuging the cells at 800 rpm for 10 min three times. Red blood cells were lysed with ACK Lysing Buffer (Gibco).

### Flow Cytometry

Surface marker staining was performed in 1×PBS at 4° C. containing the flow antibody cocktail for 30 min following 15 min blocking with human TruStain FcX (BioLegend). Cells were washed in FACS buffer (PBS with 2% FBS (ThermoFisherScientific)) and fixed in 2% paraformaldehyde in PBS (ThermoFisherScientific). For intracellular staining, cells were fixed and permeabilized using the FOXP3/Transcription Factor Staining set (eBioscience) and then incubated with intracellular antibodies at room temperature for 30 min. Samples were acquired on a LSR Fortessa (BD Biosciences) and data were analyzed using FlowJo version 10 software.

### Human ILC Sorting

Prior to sorting, isolated PBMCs were stained with human TruStain FcX (BioLegend) and incubated with the following cocktail of lineage antibodies conjugated to the FITC fluorophore: CD3 (OKT3), CD3 (UCHT1), CD4 (RPAT4), CD8a (RPAT8), CD14 (M5E2), CD15 (W6D3), CD19 (HIB19), CD20 (2H7), TCRαβ (IP26), TCROY (B1), CD33 (HIM3-4), CD34 (581), CD203c (NP4D6), FCar1a (AER37), CD79a (HM47) and CD138 (MI15). Cells are washed in FACS buffer, resuspended in EasySep Buffer (StemCell Technologies) and enriched using the EasySep FITC Positive Selection Kit II (StemCell Technologies), as per manufacturer instructions. Enriched cells were stained with a cocktail containing CD94 (PerCP-Cy5.5, DX22), NKG2D (PerCP-Cy5.5, 1D11), CD127 (PE, HIL-7R-M21), CD16 (PE-Dazzle594, 3G8), CD117 (PE-Cy7, 1D11), CCR6 (APC, G034e3), FVS700, CD45 (APC-Cy7, HI30), CRTh2 (BV421, BM16) and CD56 (BV605, HCD56) and sorted using a FACSAria Fusion (BD Biosciences).

### Human ILC Expansion

Sorted human ILC2s and ILC3s were cultured in complete X-Vivo media made of X-Vivo15 (Lonza) supplemented with 5% human AB serum (Sigma), 100U/mL Penicillin-Streptomycin (Gibco) and 1× GlutaMAX (Gibco). CD56^{bright} and CD56^{dim} NK cells were cultured in NK MACS media (Miltenyi Biotec) supplemented with 5% human AB serum. ILC2s were expanded using 100 IU/mL IL-2 (SteriMax), 20 ng/ml IL-7 and 20 ng/ml IL-33 (BioLegend). ILC3s were expanded using 100 IU/mL IL-2, 20 ng/ml IL-1β, 20 ng/ml IL-7 and 20 ng/ml IL-23 (BioLegend). CD56^{bright} and CD56^{dim} NK cells were expanded using 1000 IU/mL of IL-2, 20 ng/ml IL-15 and 20 ng/ML IL-18.

### Cytokine and Chemokine Assays

For intracellular cytokine analysis, 2×10⁵ cells were plated in a 96-well round bottom plate (Corning) and stimulated with Cell Stimulation Cocktail (eBioscience) for 6 hours with GolgiStop and GolgiPlug (BD Biosciences) added after 2 hours. Cells were then stained with surface and intracellular flow cytometry antibodies. For secreted cytokine and chemokine analysis, cells were plated in a 96-well round bottom plate in complete X-Vivo media and stimulated with 100 IU/mL of IL-2. After 16 hours, supernatant was taken from the wells and frozen at −80° C. Analytes were measured using the 12-plex LegendPlex Human Th Cytokine Panel (BioLegend) or the 13-plex LegendPlex Human Proinflammatory Chemokine Panel 1 (BioLegend) as per manufacturer's instructions.

### Xenograft GVHD Mouse Model

Six- to ten-week-old NOD.Cg-Prkdc^{scid} Il2rg^{tm1Wj}/SzJ (NSG) mice were given 150 cGy full body irradiation one day prior to intravenous tail vein injection of 1×10⁷ freshly isolated PBMCs with or without 1×10⁷ expanded ILC2s resuspended in PBS. Control mice were injected with PBS. Mice are monitored daily for symptoms of GVHD including weight loss, fur loss and skin inflammation, hunch, activity, and pain, scored on a scale of 0-3. Blood drawn at day 7, 14 and experimental or humane endpoint was centrifuged, and red blood cells were lysed with ACK Lysing Buffer (Gibco) prior to flow cytometry staining.

Splenocytes and bone marrow cells were harvested at endpoint for flow cytometric analysis. Spleens were weighed and mashed through a 70 μm filter into PBS. Cells were centrifuged and any remaining red blood cells were lysed using ACK Lysing Buffer. For bone marrow, the left and right femur bones of the mice were taken, and bone marrow cells were extracted using PBS and a 29.5G needle (BD Biosciences). Cells were passed through a 70 μm filter and centrifuged prior to flow antibody staining.

For intestinal immune cells, the colon or small intestines were flushed with 1×PBS and sliced longitudinally with a scalpel and rinsed in 1×PBS. The tissue was then cut into 1-2 cm long segments and added to 2 mM EDTA in 1×PBS and rotated on a MACSmix Tube Rotator (Miltenyi Biotec) for 30 min at room temperature. Tissue was then strained through a 70 μm filter (FisherScientific) to isolate intraepithelial lymphocytes. The remaining tissue was added to digestion buffer containing 1,650 NPA U AOF BP protease (VitaCyte), GMP Grade (VitaCyte), 2,500 CDA U Collagenase MA, GMP Grade (VitaCyte) and 100 ug/mL DNaseI (StemCell Technologies) in Hank's Balanced Salt Solution with calcium (HBSS, Wisent Bio Products), in a 1.5 mL microcentrifuge tube (Eppendorf). Tissue was then teased apart using sterile scissors, transferred to a 50 ml conical tube (FisherScientific) then rotated using MACSmix Tube Rotator (Miltenyi) at 37° C. for 30 min. Tissue was mashed through a 70 μm filter and washed in RPMI (Gibco) with 5% FCS twice.

### Histology

Harvested spleen, lungs, liver and colon were fixed for 3 days in 10% neutral buffered formalin and stored in 70% before paraffin embedding. Paraffin embedding, tissue slicing, H&E staining and immunohistochemical staining was performed by the UHN Pathology Research Program Laboratory. Quantification of cells expressing IHC markers was done using the HALO Image Analysis Platform and reported as a percent of all cells identified.

### Co-Cultures

Naïve CD4⁺ and total CD8⁺ T cells are isolated from the blood of healthy donors using the EasySep Human Naïve CD4⁺ T cell isolation kit (StemCell Technologies) or the EasySep Human CD8⁺ T cell isolation kit (StemCell Technologies). Cells are plated in a 96-well flat bottom plate at 5×10⁴ per well. T cells are stimulated with human CD3/28 T-Activator DynaBeads (Gibco) at a ratio of 1 bead:8 T cells. Expanded ILC2s are washed with PBS three times and added to the T cells at a ratio of 1:1. 72 hours later, T cells are stained for intracellular markers using flow cytometry. For supernatant cultures, expanded ILC2s are plated in a 96-well round bottom plate at a density of 5×10⁵ cells/mL in completed X-Vivo media without cytokines. After 16 hours, the supernatant is harvested, centrifuged at 1500 rpm for 10 min to eliminate remaining cells and frozen at −80° C. For T cell cultures, supernatant is thawed and added to T cells at a 1:1 ratio. Fresh supernatant is added every 24 hours for 72 hours.

For inhibitor experiments, the following antibodies were added at 10 ug/mL to inhibit cytokine signaling: UltraLeaf anti-human IL-4, UltraLeaf anti-human IL-9, UltraLeaf anti-human IL-10 and UltraLeaf anti-human IL-13 (BioLegend). ARL67156 (Tocris) and PBS12379 (Tocris) were added at 1 μg/mL to inhibit activity of CD39 and CD73 respectively. Inhibitors were added at day zero and every 24 hours for 72 hours.

### Single Cell CITE Sequencing

1×10⁶ expanded ILCs were washed twice in 1× Cell Staining Buffer (BioLegend) and incubated in TruStain FcX for 15 min. Cells were incubated with a cocktail of TotalSeq-C antibodies (BioLegend) for 30 min. Cells were then washed three times in 1× Cell Staining Buffer and resuspended in 1×PBS with 0.04% BSA (Millipore Sigma). Samples were prepared sequencing using the 10× Genomics Single Cell 5′ v2 platform in accordance with manufacturer's instructions. 12,000 cells were added to the Chromium Next GEM Chip K and loaded onto the 10× Chromium Controller (10× Genomics). Reverse transcription, cDNA amplification and sequencing libraries using the 10× Genomics Single Cell 5′ v2 reagents. Samples were sequenced to a depth of 40,000 reads. Read alignment to the reference human genome (GRCh38/hg38) and gene expression matrices were generated using CellRanger version 3.1.0.

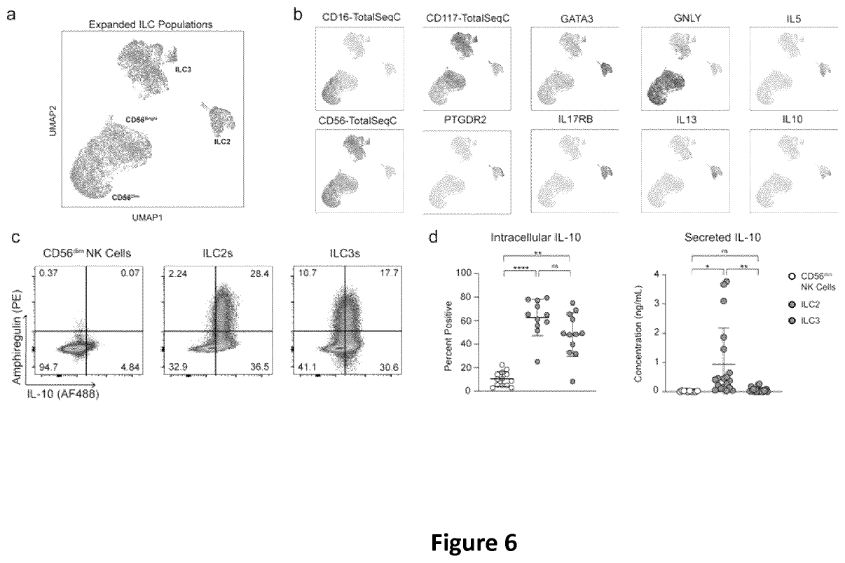

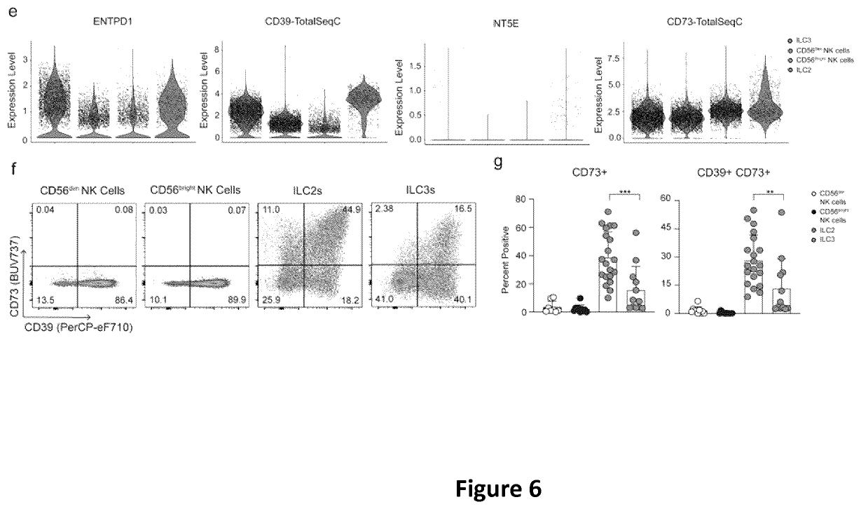

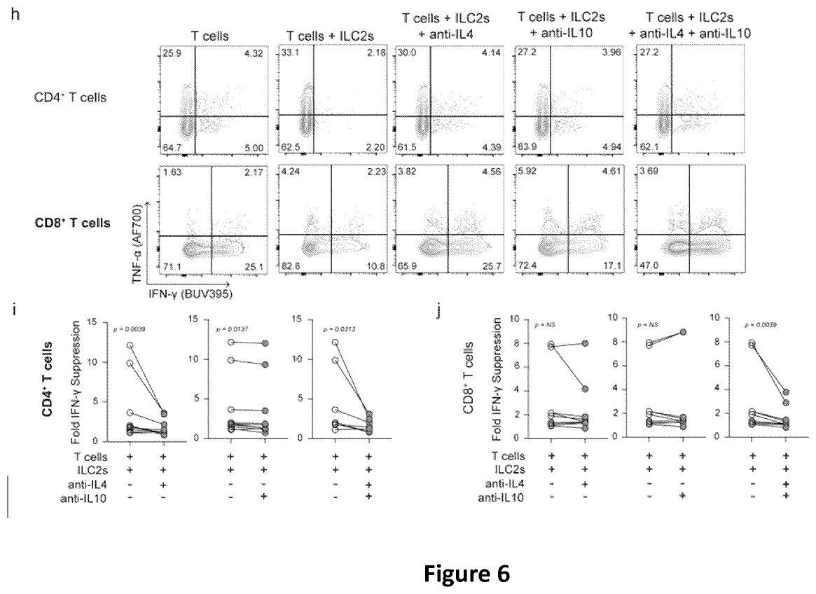

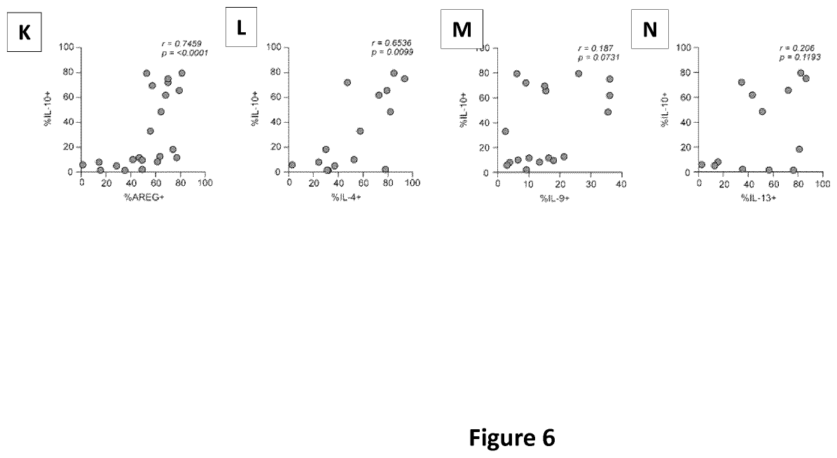

Single cells were filtered to exclude cells that expressed >10% mitochondrial content, <1000 total transcripts and <200 unique genes. Data was log normalized, principal component analysis was preformed, and cells were clustered with the top 20 principal components with the Louvain community algorithm using Seurat's FindNeighbors and FindClusters⁴⁶. Cell clusters were visualized using the Uniform Manifold Approximation and Projection (UMAP)⁴⁷. Clusters were assigned to ILC subsets based on expression of hashtag antibodies, lack of lineage markers and expression of subset associated markers (FIG. 6a). Cluster defining markers were identified using FindMarkers in Seurat.

### Statistics

Statistical significance was determined by Kruskal-Wallis test or one-tailed Mann-Whitney test. The log-rank (Mantel-Cox) test was used for Kaplan-Meir curves. Analysis of correlational data was calculated using Spearman correlation. The number of replicates is represented by n and is shown in each figure legend. * p<0.05; ** P<0.01; *** P<0.001; **** P<0.0001; ns, not significant. Data analysis was preformed using GraphPad Prism v9.

### Results and Discussion

ILC2s in Peripheral Blood Differentiate HSCT Patients with or without GVHD

HSCT is often a curative therapy for hematological malignancies, however, up to 80% of patients develop aGVHD characterized by donor T cells attacking recipient tissues including the skin, liver, lungs, and gastrointestinal tract²¹. Prior groups have reported increased numbers of CD56^{bright} NK cells, ILC2s and ILC3s in peripheral blood of HSCT patients that do not develop GVHD compared to those that do¹¹. How that correlated with differences in T cell populations that contribute to GVHD pathology was unclear.

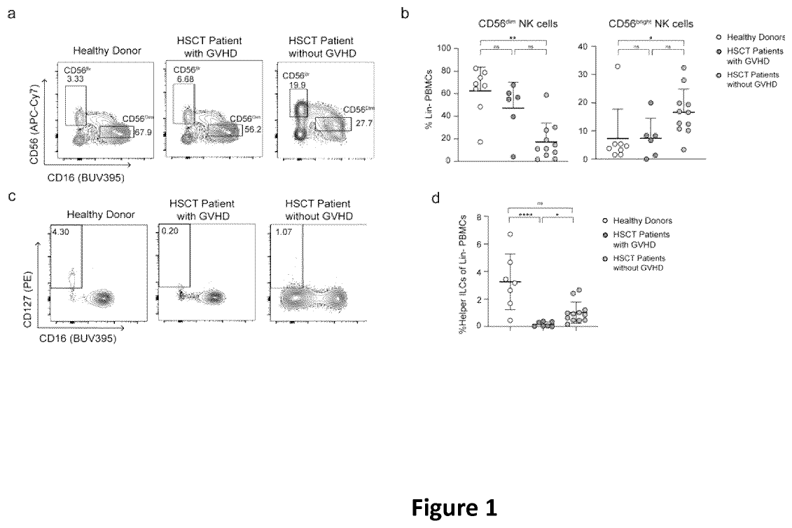

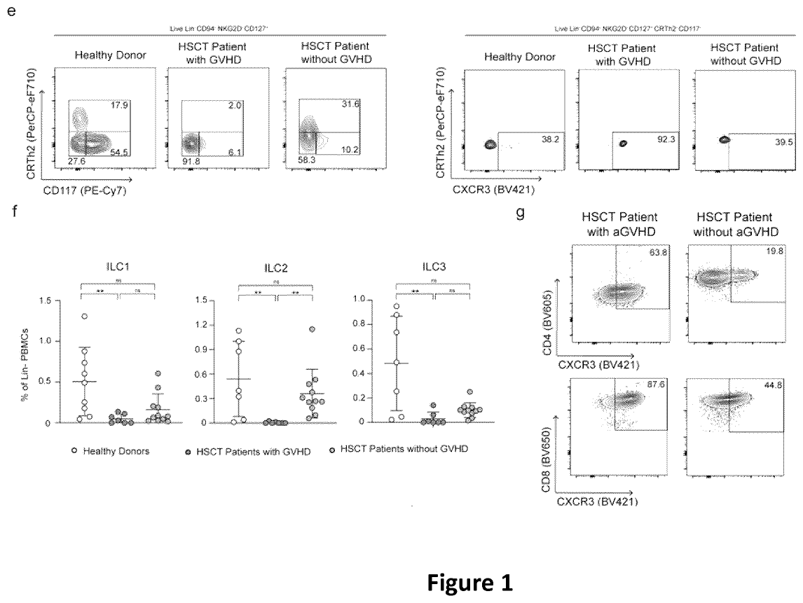

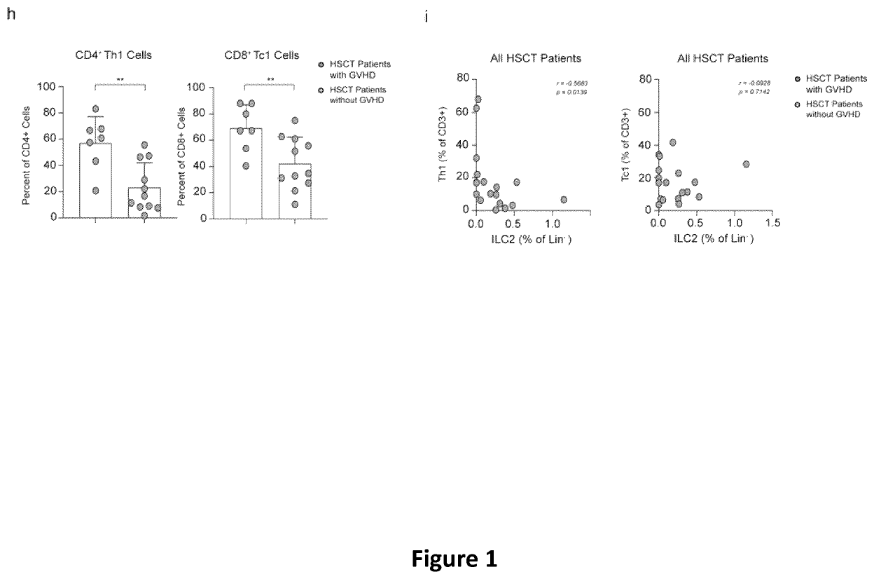

We analyzed the proportion of different ILC subsets and CD4⁺ and CD8⁺ T cell subsets in the blood of HSCT patients at the time of diagnosis with aGVHD and compared them to HSCT patients without aGVHD and healthy donors. In line with previous literature, patients who did not develop aGVHD had decreased proportions of CD56^{dim} NK cells and increased proportions of CD56^{bright} NK cells compared to both patients with aGVHD and healthy donors^{9,10} (FIG. 1a,b). Similarly, patients that developed aGVHD had a reduced proportion of helper ILCs (Lin⁻ CD127⁺) compared to both healthy donors and HSCT patients without aGVHD (FIG. 1c,d). This reduction in helper ILCs was evident across all three subsets (ILC1s, ILC2s and ILC3s) compared to healthy controls (FIG. 1e,f). In contrast, HSCT patients without aGVHD had significantly higher levels of ILC2s, but no statistically significant difference in ILC1s or ILC3s when compared to patients with aGVHD (FIG. 1e,f).

CXCR3 is also a well-known marker for CD4⁺ T helper type 1 (Th1) and CD8⁺ type 1 cytotoxic T (Tc1) cells²²⁻²⁴ and have been linked in preclinical GVHD models to aGVHD^{25,26}. Over the course of assessing ILCs in HSCT recipients, we were also monitoring differences in proportions of Th1 and Tc1 cells by examining CXCR3 expression on CD4⁺ and CD8⁺ T cells. HSCT patients with aGVHD had increased percent of Th1 and Tc1 cells as a proportion of total CD4⁺ and CD8⁺ T cells, respectively (FIG. 1g,h). Of note, a significant increase in Th1 cells but not Tc1 cells as a percent of all CD3⁺ T cells strongly correlated with low proportions of ILC2s across all HSCT patients (FIG. 1i). This inverse relationship between ILC2s and T cells was of note, as ILC2s have been shown to regulate T cell responses in diverse contexts¹⁵. To assess whether ILC2s could have similar protective functions in HSCT, we examined whether adoptive transfer of human ILC2s could protect in a xenograft model of GVHD.

### Human ILC2s Rapidly Expand In Vitro and Maintain Expression of Signature Cytokines

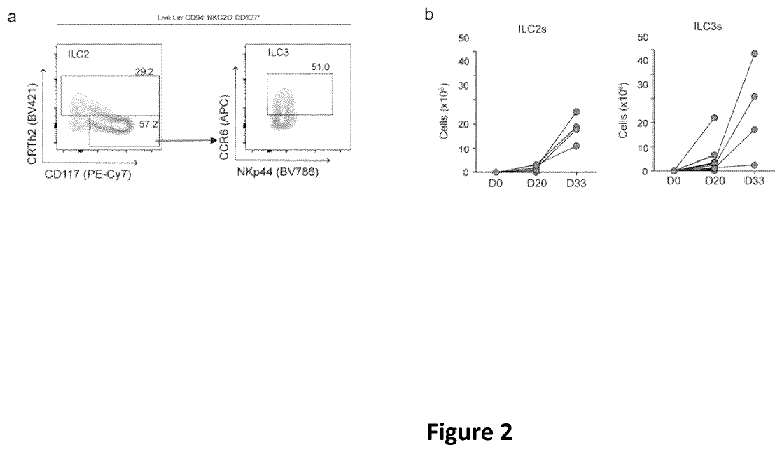

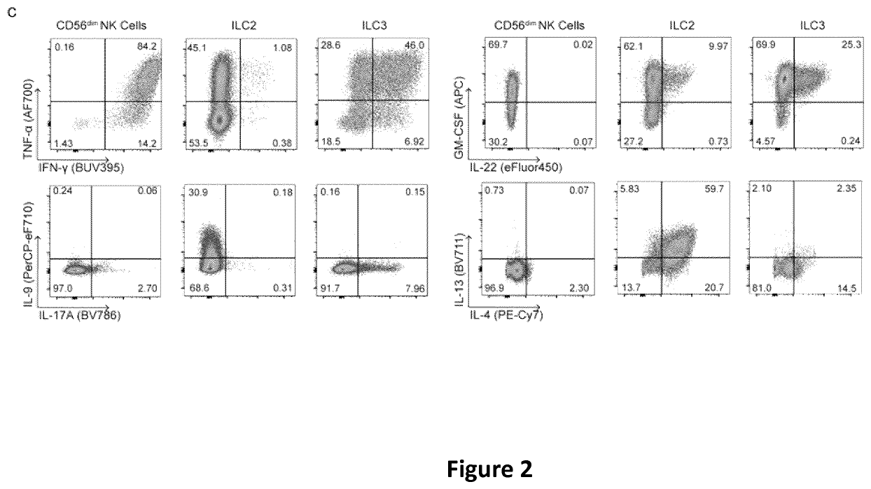

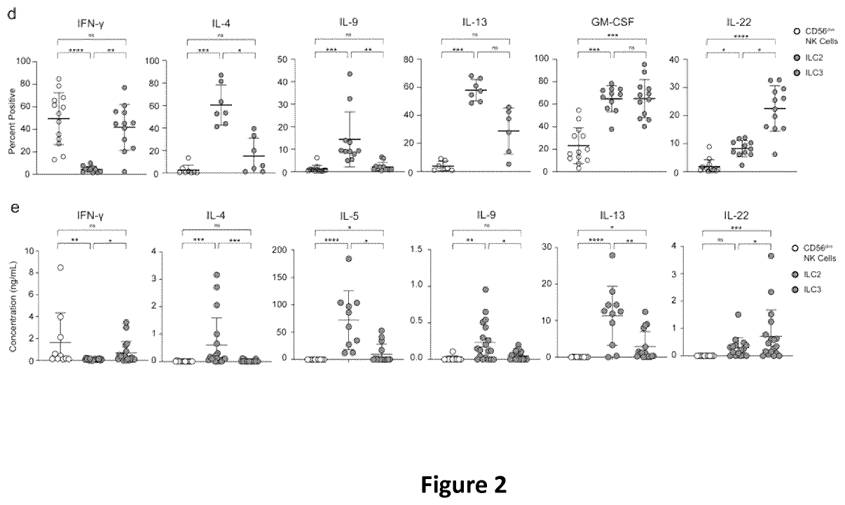

To examine potential protective functions of human ILC2s in a xenograft GVHD model, we first had to develop methods to isolate and expand low abundance ILC2s from human blood in a manner that maintains expression of signature cytokines. We used a combination of ILC2-associated markers to isolate ILC subsets from healthy donor PBMCs using fluorescence activated cell sorting (FACS), along with sorting CD56^{dim} NK cells and ILC3s as a control. Helper ILCs were first gated as Live, Lineage CD94 NKG2D CD127⁺. ILC2s were then isolated based on being CRTh2⁺ CCR6⁻ CD56⁻ and ILC3s as CRTh2 CD117⁺ CCR6⁺ (FIG. 2a). CD56^{dim} NK cells were sorted for low expression of CD56, high expression of CD16⁺ and negative for CRTh2 and CCR6.

ILC2s and ILC3s were then expanded in vitro using complete X-VIVO 15 medium supplemented with IL-2, IL-7, IL-33 or IL-2, IL-7, IL-1β, and IL-23, respectively. NK cells were cultured in NK MACS medium containing IL-2, IL-15, and IL-18. Cell numbers were assessed at day 21 and day 33. Of note, despite being low proportion in peripheral blood, human ILC2s and ILC3s exhibited robust expansion (2.31×10⁴+7.23×10³ and 9.80×10³+3.58×10³ fold expansion respectively) after 34 days in culture (FIG. 2b).

To ensure expanded ILC2s maintained expression of signature cytokines, we performed intracellular flow cytometry staining for NK-cell cytokines (IFN-γ and TNF-a), ILC2 cytokines (IL-4, IL-9, IL-13), ILC3 cytokines (IL-22, IL-17A) as well as GM-CSF which can be expressed by several ILC subsets²⁷. Post-expansion, ILC2s strongly co-expressed IL-4 and IL-13 as well as IL-9 and GM-CSF with weak to no expression of IFN-γ, IL-17A or IL-22 (FIG. 2c, d). In contrast, CD56^{dim} NK cells strongly co-expressed IFN-γ and TNF-α and ILC3s expressed IL-22 and GM-CSF, with some donors displaying low level of IL-17A expression (FIG. 2c, d). Quantitative cytokine production was also assessed by examining secreted cytokines from ILCs using cytometric bead array (CBA) following stimulation of ILC subsets with IL-2 for 16 hours. Analysis of secreted cytokines confirmed ILC2s produced IL-4 and IL-9 with very high amounts of IL-5 and IL-13, whereas NK cells and ILC3s did not produce significant amounts of these cytokines and produced their canonical cytokines (FIG. 2e). Thus, isolated and expanded ILC2s maintained expression of subset-defining cytokines.

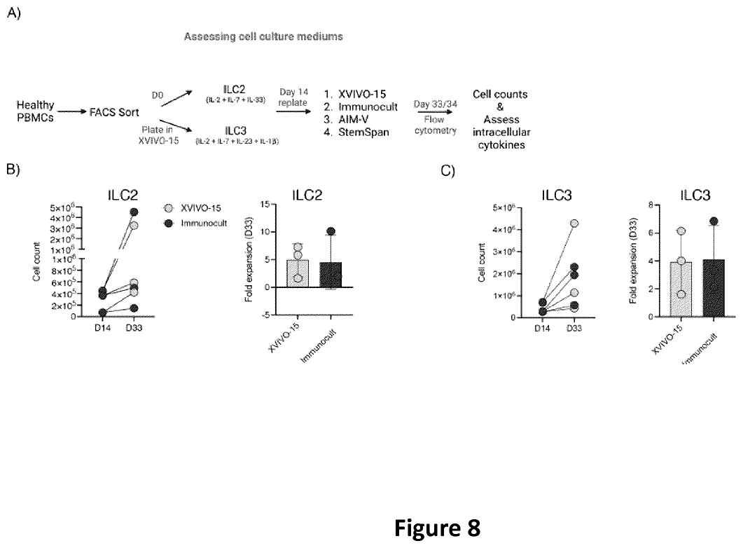

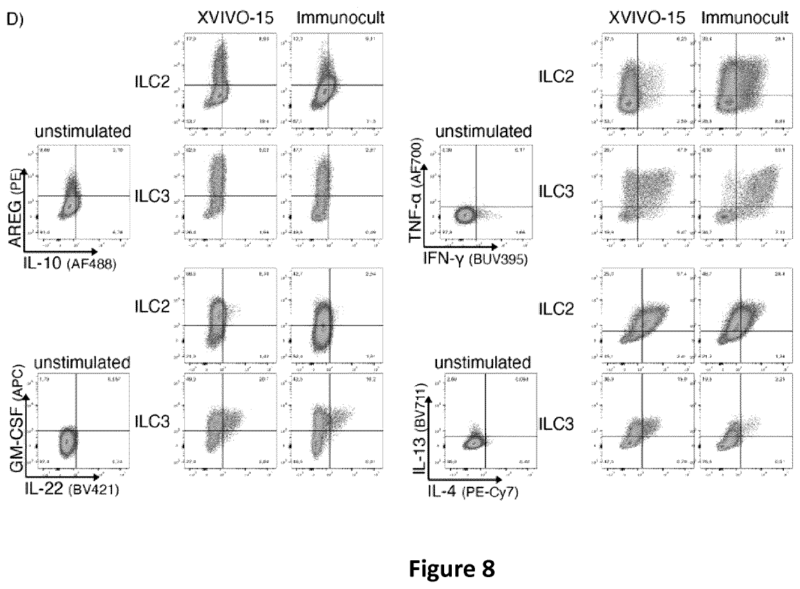

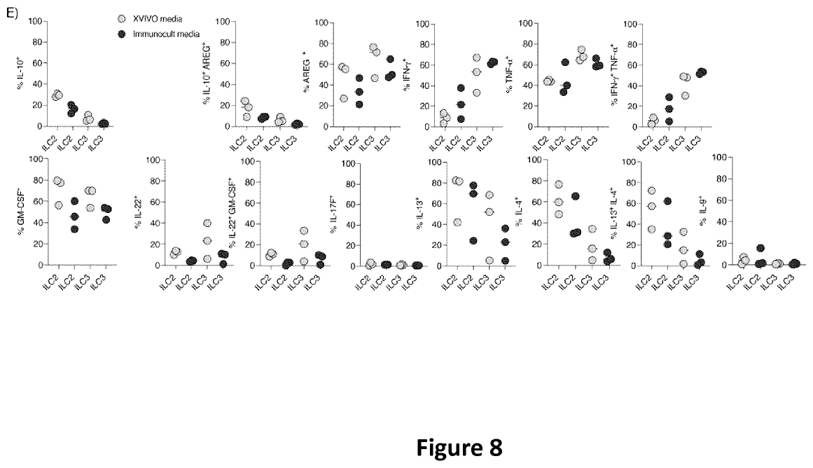

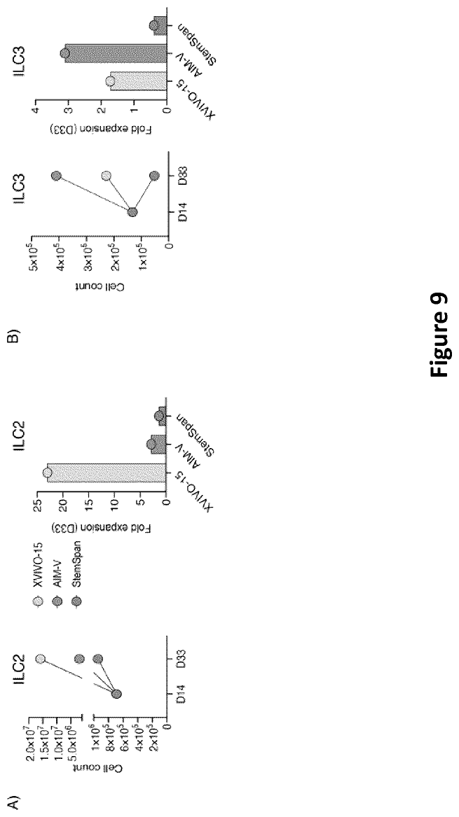

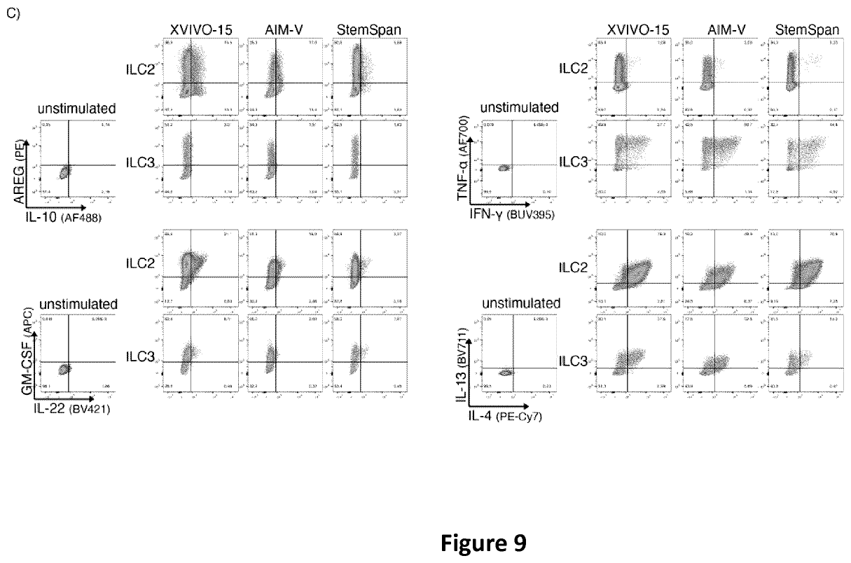

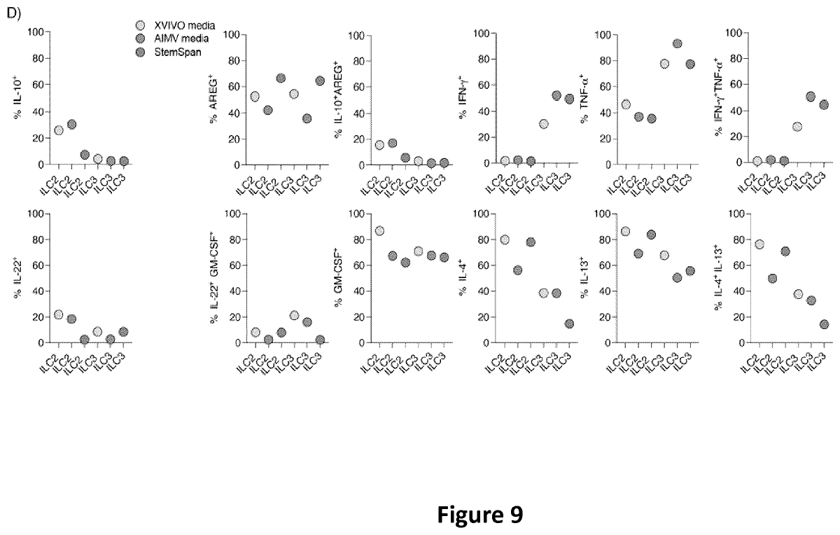

In this regard, it is notable that applicant also compared ILCs cytokine production and expansion using different GMP compatible media and surpassingly found that XVIVO-15 medium was superior to Immunocult, AIM-V and StemSpan. Specifically, XVIVO-15 was far better at providing the IL-10 phenotype for ILCs. (FIG. 8 and FIG. 9).

### Adoptive Transfer of Allogeneic Human ILC2s Limits Xenograft GVHD

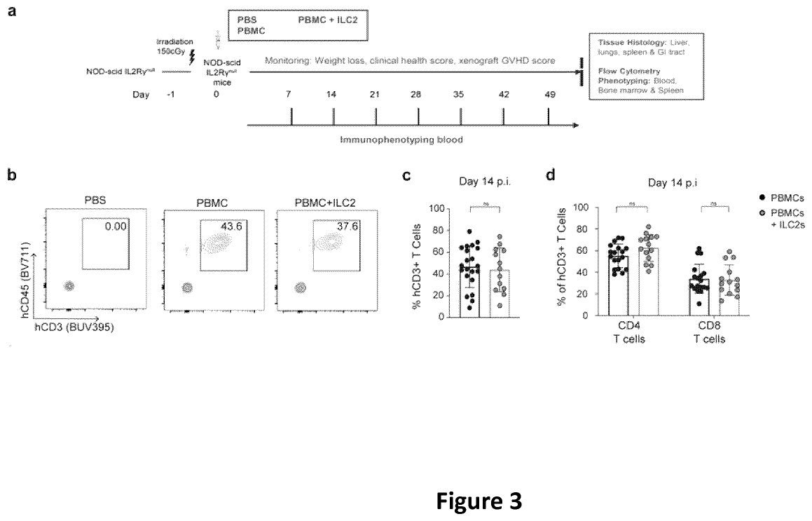

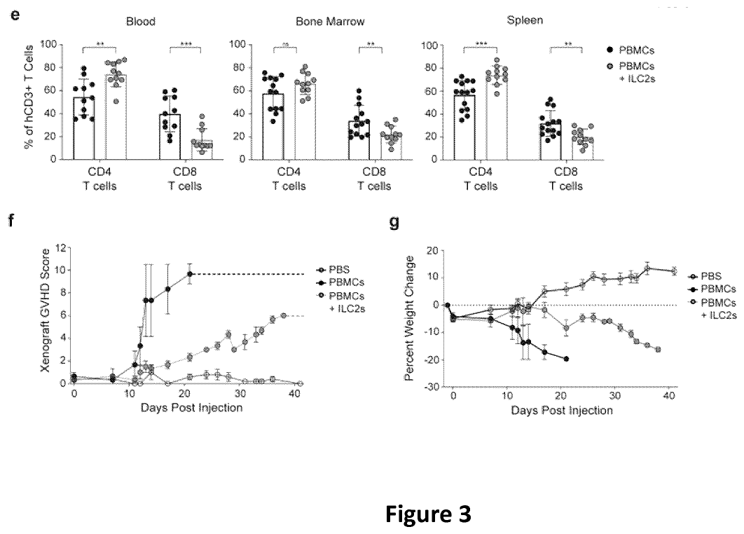

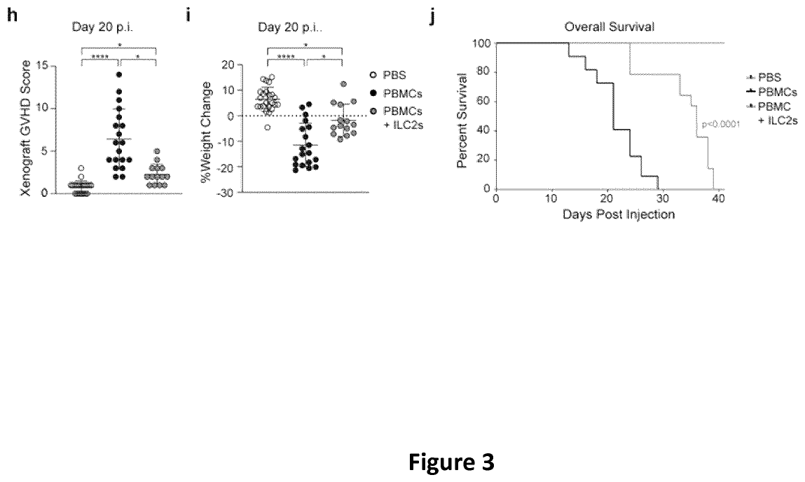

To test whether human ILC2s could have beneficial applications for aGVHD, we used a xenograft GVHD mouse model, in which 6-8-week-old NOD-scid-IL2Rg^{null} mice are irradiated and injected with human PBMCs to induce multiorgan tissue pathology similar to aGVHD after HSCT²⁸. We tested whether adoptive transfer of expanded human ILC2s could protect from xenograft GVHD by treating mice given PBMCs with expanded allogeneic ILC2s (FIG. 3a). This approach mirrors using recipient derived-ILC2s or 3^{rd} Party ILC2s which would have benefits from a cell manufacturing perspective. We also wanted to assess whether the addition of ILC2s would limit allogeneic T cell engraftment into the mice. After 14 days, strong engraftment of human CD45⁺ CD3⁺ T cells was observed in the peripheral blood of the mice, with no significant difference in T cell proportions between mice given PBMCs alone and mice treated with ILC2s (FIG. 3b, c). There was also no significant alterations in the proportion of CD4⁺ and CD8⁺ T cells in the blood 14 days post injection (FIG. 3d), however at endpoint there was a moderate increase in the proportion of CD4⁺ T cells and a reciprocal decrease in CD8⁺ T cells respectively, in the blood, bone marrow and spleen of mice treated with ILC2s (FIG. 3e).

Mice were monitored for signs of xenograft GVHD using weight loss and a composite xenograft GVHD score, measuring fur loss, skin inflammation, hunch, activity, and pain. Mice treated with ILC2s had a delayed onset of GVHD symptoms and a reduction in weight loss (FIG. 3f, g). At day 20 post-injection, a time point when approximately half of the mice given PBMCs alone were alive and mice treated with ILC2s were all alive, the mice treated with ILC2s had a significantly lower xenograft GVHD score and displayed lower weight loss compared to mice given PBMCs alone across three independent experiments with female mice (FIG. 3h, i). There were similar trends in parallel experiments in male mice, although the kinetics of GVHD differed between male and female mice. This single infusion of ILC2s not only reduces GVHD severity but also significantly improves survival outcomes of the mice (FIG. 3j). Taken together, these experiments support human ILC2s limit pathology and enhance overall survival in this model of GVHD.

Allogeneic ILC2s Regulate CD4⁺ and CD8⁺ T Cells In Vivo in Xenograft GVHD

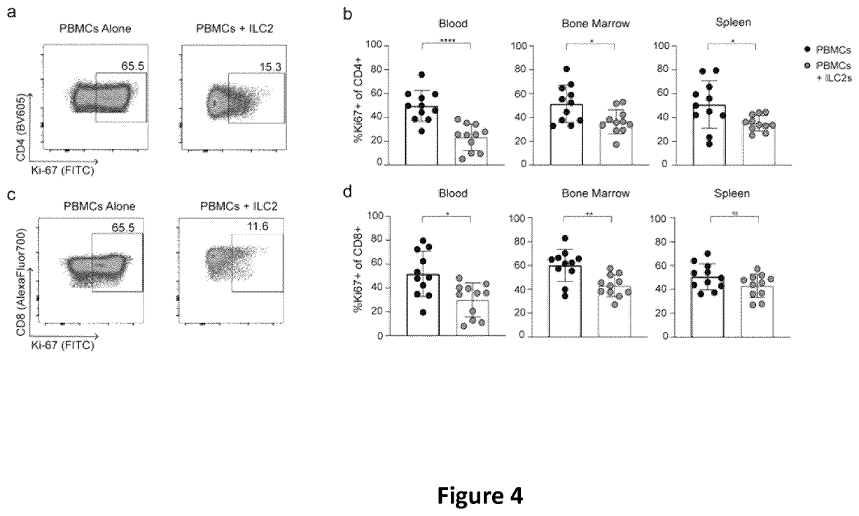

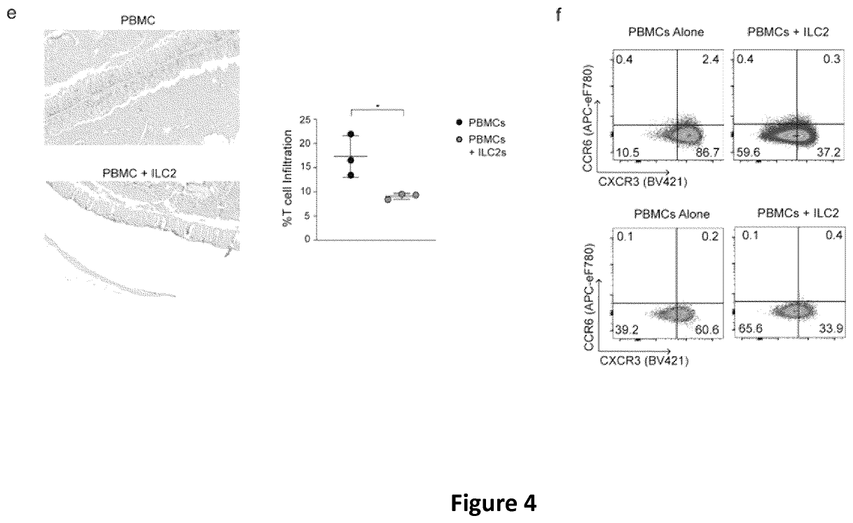

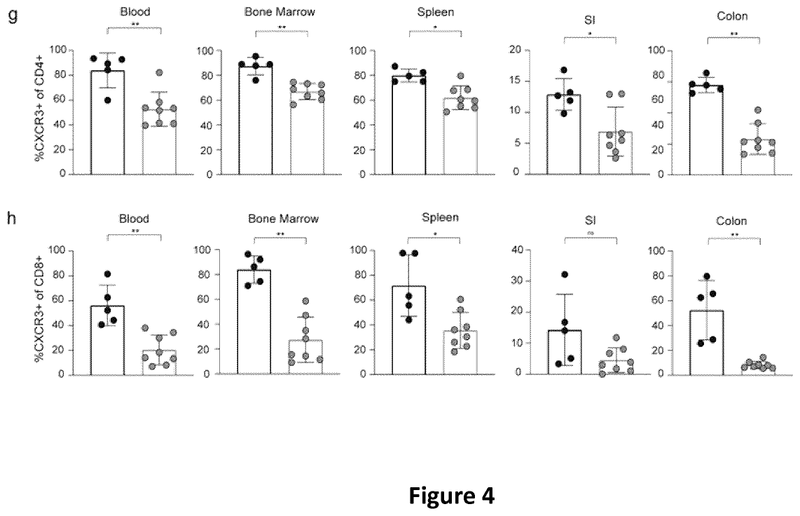

We next sought to determine how ILC2s were protecting from GVHD. As this model is predominantly a T cell model, with very few non-T cells engrafting post-PBMC transfer, we examined whether adoptively transferred ILC2s were directly regulating allogeneic T cell responses in vivo. We looked at T cell expression of Ki-67, a marker of cellular proliferation, to examine the ability of the ILC2s to affect T cell expansion. Both CD4⁺ and CD8⁺ T cells had a reduced Ki-67 expression in the blood, bone marrow and spleen of mice treated with ILC2s (FIG. 4a,b), supporting that proliferation of both cytotoxic and helper T cells is inhibited with ILC2 administration.

While T cell proliferation was inhibited with ILC2 co-administration, no significant differences in PD-1, CTLA-4 and CD25 were observed by CD4⁺ or CD8⁺ T cells in the blood, bone marrow and spleens of NSG mice treated with ILC2s.

The gastrointestinal tract is one of the primary areas affected in aGVHD, a phenotype which is also observed in the xenograft GVHD model. We examined T cell infiltration into the colons of NSG mice treated with PBMCs alone or those that also received ILC2s. CD3 immunohistochemistry staining revealed a stark reduction in the amount of T cell infiltration into colons of ILC2-treated mice but not in the lungs, spleen, or liver (FIG. 4e, f).

Previous research has indicated that CXCR3 expression on T cells in GVHD models drives trafficking to GVHD target organs, including the skin and intestines^{29,30}. CXCR3 also marks CD4⁺ Th1 and CD8⁺ Tc1 cells²²-24 which have been shown to drive intestinal damage in aGVHD models and correlate with disease in human patients^{31,32} As we had observed an inverse relationship between ILC2s and Th1 and Tc1 cells (FIG. 1g,i), we therefore asked whether ILC2s were influencing T cell subsets within the xenograft GVHD model. We examined CXCR3 expression on T cells in the blood, bone marrow, spleen, colon and small intestine of PBMC-treated NSG mice that received allogeneic ILC2s or did not. In all compartments, mice infused with allogeneic ILC2s had reduced expression of CXCR3 on CD4⁺ and CD8⁺ T cells (FIG. 4g-j). While CXCR3 expression has been identified as a marker of Th1 cells and Tc1 cells, Th2 and Th17 cells express CRTh2 and CCR6 respectively. Despite seeing decreased CXCR3 expression, no increase in CRTh2 or CCR6 was observed, indicating adoptive transfer of ILC2s did not promote differentiation to other T cell subsets but instead inhibited Th1 and Tc1s development or activity (FIG. 4g-j) 23,24,33

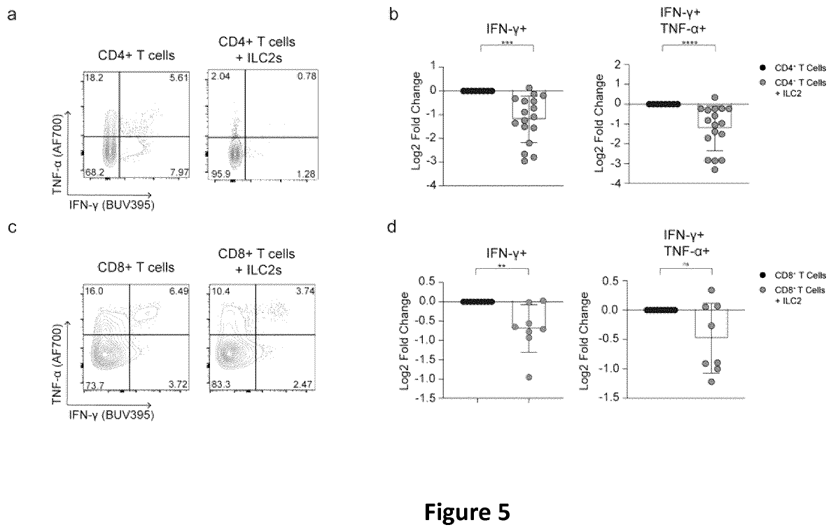

To support ILC2-mediated regulation was due to direct effects on T cells, in vitro co-cultures were performed with ILC2s and allogeneic CD4⁺ or CD8⁺ T cells. When the ILC2s were cultured with allogeneic naïve CD4⁺ T cells, there was a dramatic reduction in IFN-γ+ T cells as well as reduced co-expression IFN-γ and TNF-α after 3 days in culture (FIG. 5a,b). Decreased IFN-γ was also observed when allogeneic ILC2s were cultured with CD8⁺ T cells (FIG. 5c,d).

The addition of an IL-4-neutralizing antibody strongly reduced ILC2₁₀-mediated suppression of IFN-γ by CD4⁺ T cells in vitro, but blocking IL-9, IL-13 or CD39/CD73 had no effect (FIG. 5E,F). Neutralizing IL-10 also decreased suppression of CD4⁺ T cell-IFN-γ, but to a lesser extent than IL-4 (FIG. 5E,F). In contrast, the combination of anti-IL-4 and anti-IL-10 abrogated ILC2₁₀-mediated suppression of IFN-γ by CD8⁺ T cells, which was not observed with anti-IL-4 or anti-IL-10 alone (FIG. 5E,G). Thus, IL-4 and IL-10 underlie ILC2₁₀ ability to directly suppress allogeneic CD4⁺ and CD8⁺ T cells cytokine production, with differing contributions of IL-4 and IL-10 to the regulation of CD8⁺ and CD4⁺ T cells

### Identifying Molecular Mechanisms ILC2s May Use to Regulate Allogeneic T Cells

To identify potential mechanisms by which ILC2s were regulating allogeneic T cells, we preformed single cell Cellular Indexing of Transcriptomes and Epitopes by Sequencing (scCITE-Seq) on expanded ILC2s, ILC3s, and both CD56^{dim} and CD56^{bright} NK cells. We were interested in identifying immunoregulatory molecules, cytokines and other suppressive molecules uniquely expressed by expanded ILC2s. Expanded CD56^{dim} NK cells, CD56^{bright} NK cells, ILC3s and ILC2s were labelled separately with hashtag antibodies and stained with a TotalSeq-C antibody cocktai). The hashtag antibodies allowed for easy identification of clusters corresponding to each of the expanded ILC subsets (FIG. 6a). Importantly, all ILC subsets examined expressed lineage defining markers that differentiated them other ILC family members. Both CD56^{bright} and CD56^{dim} NK cells expressed GNLY and CD56, while only the CD56^{dim} NK cells expressed CD16. ILC3s strongly expressed CD117 and ILC2s expressed PTGDR2, GATA3, IL17RB, IL13 and IL5 (FIG. 6b). Intriguingly, one of the top genes identified as being uniquely expressed by ILC2s was IL-10 (FIG. 6b). Using intracellular cytokine staining, we validated that expanded ILC2s expressed high proportions of IL-10 at the protein level (FIG. 6c,d). We also analyzed previously acquired expanded ILC supernatant and found high levels of IL-10 secretion primarily in the expanded ILC2s (FIG. 6c,d). While expanded ILC3s also had high expression of IL-10 by intracellular flow cytometry after PMA-lonomycin stimulation, they lacked IL-10 secretion with cytokine alone stimulation, indicating that they may have the potential to produce IL-10 but only in particular contexts. The high IL-10 expression by expanded ILC2s was of note, as several recent studies identified IL-10 producing ILC2s as having immunoregulatory properties with potential clinical applications^{16,20,34} and both IL-2 and IL-33 which were used to expand ILC2s in this study have been linked to promoting IL-10 production in ILC2s^{16,20}.

After identifying expression of IL-10, we also examined expression of other molecules linked to regulatory cells such as CD4⁺FOXP3⁺ Tregs and T regulatory 1 (Tr1) cells. We found a low or background level of expression of checkpoint molecules such as PD-1, PD-L1, CTLA-4 or LAG3 at the gene and protein level, which are associated with Tr1 cells^{35,36}. Additionally, we did not detect IL12A and EBI3 RNA, which make up the regulatory cytokine IL-35. Despite ubiquitous transcriptional expression of TFGB1 across ILC subsets, there was no protein level expression of active TFG-β1 detected in NK cells, ILC2s or ILC3s expanded using our approach). While these regulatory molecules expressed by Tregs and Tr1 cells were absent, both expanded ILC2s and ILC3s expressed ENTPD1, the gene for CD39, as well as expressed CD39 at the protein level which was not strongly seen in either NK cell subset (FIG. 6e). ILC2s and ILC3s weakly expressed NT5E (CD73), however only ILC2s and CD56^{bright} NK cells exhibited surface CD73 protein expression (FIG. 6e). This is in line with prior reports that ILC3s and IL-33 stimulated ILC2s can dephosphorylate ATP and AMP into the immunosuppressive molecule adenosine by expression of the ectoenzymes CD39 and CD7313,37. Flow cytometry analysis verified the expression of these enzymes on expanded ILCs, with ILC2s co-expressing high levels of both CD39 and CD73 (FIG. 6f, g). This suggested ILC2s might have the capacity to fully dephosphorylate ATP to immunosuppressive adenosine which can inhibit the functional activity of both CD4⁺ and CD8⁺ T cells^{38,39}.

Expanded ILC2s Directly Regulate CD4⁺ and CD8⁺ T Cell Cytokine Expression In Vitro Through IL-4 and IL-10

Having confirmed direct ability to suppress Th1 and Tc1 cells, we next sought to determine the mechanisms by which ILC2s regulated allogeneic T cells. To rule out cell contact-dependent mechanism and assess if ILC2-regulation of T cells was due to secreted factors, we treated allogeneic T cells with supernatants from IL-2-stimulated expanded ILC2s. Addition of supernatants of expanded ILC2s to naïve T cells alone were equally able to suppress T cell responses compared to the addition of ILC2s themselves. We therefore examined whether inhibition of secreted factors produced by expanded ILC2s would abrogate their immunoregulatory functions.

ILC2s were cultured with allogeneic T cells in the presence of neutralizing antibodies or inhibitors to molecules of interest identified by CITE-seq (IL-10, CD39, CD73) as well as cytokines produced by ILC2s that were previously linked to T cell regulation (IL-4, IL-9, IL-13) 40-42. The addition of an IL-4-neutralizing antibody strongly reduced ILC2-mediated suppression of IFN-γ on CD4⁺ T cells but blocking IL-9 and IL-13 or inhibiting CD39 and CD73 had no effect on this suppression (FIG. 6h,i). Neutralizing IL-10 alone significantly decreased suppression of CD4⁺ T cell IFN-γ but to a lesser extent than IL-4 alone (FIG. 6h,i). In contrast, the combination of both anti-IL4 and anti-IL-10 antibodies to ILC2-CD8⁺ T cell co-cultures limited the effectiveness of ILC2s to suppress IFN-γ expression, which was not observed with addition of anti-IL-4 or anti-IL-10 alone (FIG. 6h,j). Thus, ILC2-derived IL-4 and IL-10 underlie IL-10 producing ILC2s suppression of allogeneic CD4⁺ and CD8⁺ T cells, with differing contributions of IL-4 and IL-10 to the regulation of CD8⁺ cytotoxic and CD4⁺ helper T cells.

IL-10 expression on ILC2s was correlated with co-expression of IL-4, as well as amphiregulin (AREG) but had no significant correlation to expression levels IL-9 or IL-13 cytokines (FIG. 6k-n). High amphiregulin was also notable, as it is a key tissue reparative effects (13, 19, 20), and was linked to ILC2s protective effects in limiting GVHD pathology in a murine model, supporting expanded ILC2₁₀ could have dual regulatory and reparative potential.

The GVL effect is critical for success of HSCT for treating hematological malignancies. As we had observed direct regulation of T cell responses by ILC2 in xenogeneic GVHD, and increased ILC2 proportions in patients protected from GVHD development, a key question was whether ILC2s would have detrimental effects T cell-mediated GVL effects. Within our clinical cohort, we assessed circulating ILC2 proportions and how they correlated with relapse status. No significant differences in ILC2s proportions was observed in the blood of patients which went on to experience relapse of their malignancy with our sample size, suggesting that the increased proportion of ILC2s were not inhibiting the GVL effects (FIG. 6A).

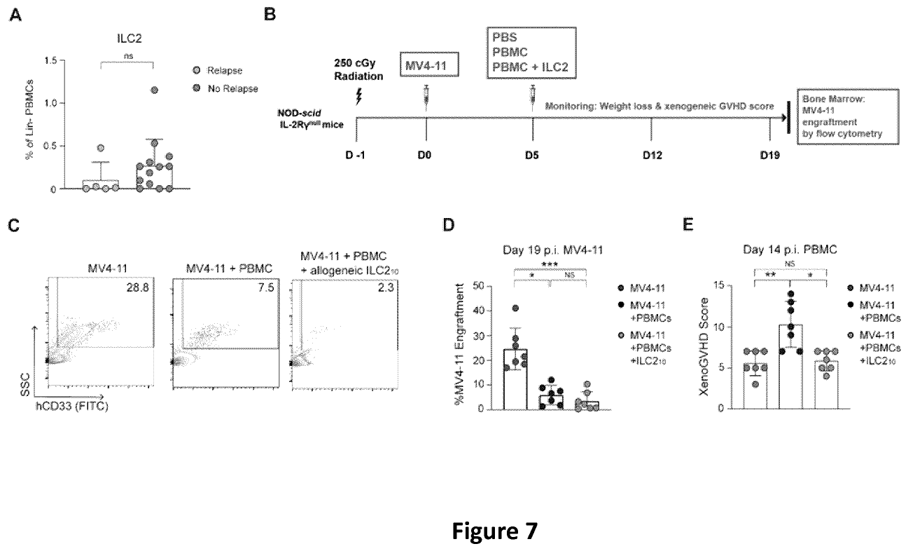

We next assessed whether ILC2₁₀ cell therapy would negatively impact T cell-mediated GVL-effects in a in a humanized mouse model. Briefly, MV4-11 cells, an acute myeloid leukemia cell line, were injected into mice 5 days prior to transfer of PBMCs alone or PBMCs with allogeneic expanded human ILC2₁₀ (FIG. 7b). The ability of T cells to kill MV4-11 cells was then assessed at day 14 following PBMC transfer. As expected, PBMC injection resulted in a reduction in MV4-11 cells in the bone marrow compared to mice receiving MV4-11 cells alone (FIG. 7c,d). Mice treated allogeneic ILC2₁₀ had comparable reductions in MV4-11 cells within the bone marrow to that observed with mice receiving PBMCs alone (FIG. 7c,d), supporting ILC2₁₀ were not impeding T cell-mediated GVL responses. Importantly, within the same mice, ILC2₁₀ treatment reduced xenoGVHD symptoms similar to what was observed without MV4-11 transfer (FIG. 7e), clearly showing simultaneous protection from pathogenic T cell responses that underlie GVHD without impairing T cell-mediated GVL effects. Collectively our findings support ILC2₁₀ do not limit the anti-leukemic effect of allogeneic T cells despite protecting from GVHD, therefore represent a strong candidate for cell-based therapies for HSCT.

### ILCs Suppress Antigen-Specific Islet Rejection and Regulate Harmful T Cell Subsets.

We found that innate lymphoid cells (ILCs) were present within healthy islets and pancreas, allogeneic ILCs were not cytotoxic to β-cells or α-cells in human islets, and ILCs do not inhibit engraftment when transplanted in vivo with human islets (data not shown).

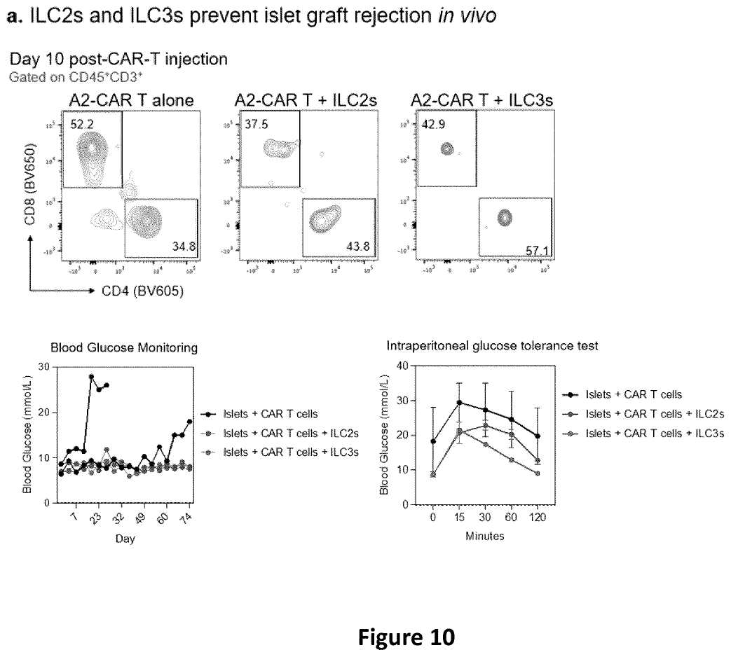

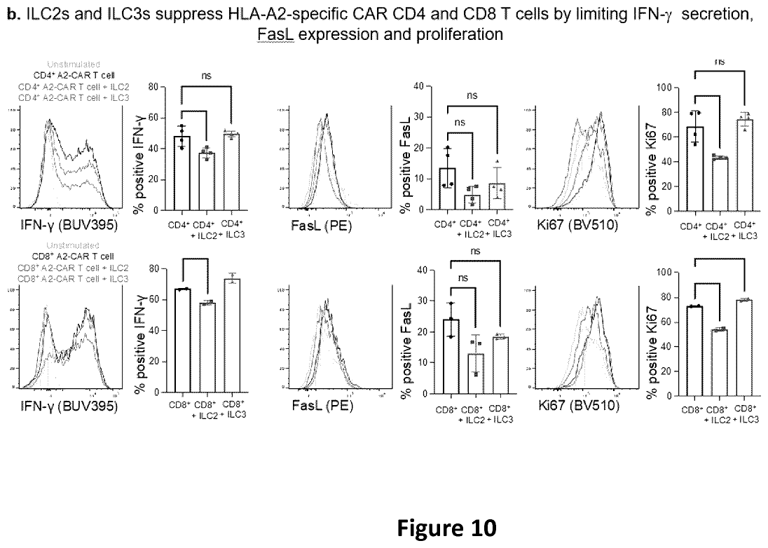

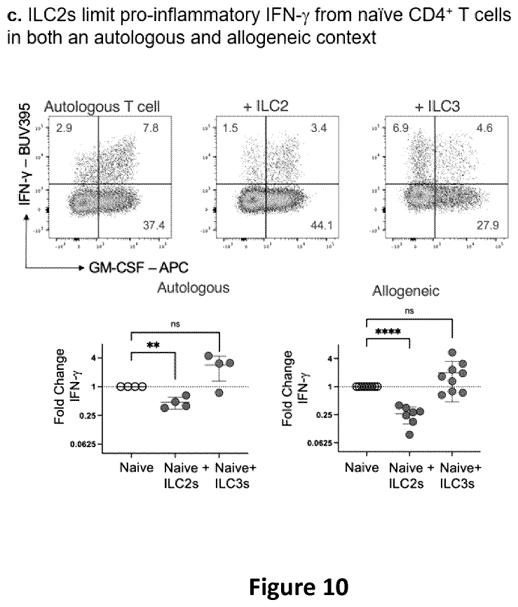

NSG mice were injected with 150 mg/kg STZ to kill endogenous β cells and transplanted with 750 HLA-A2+ IEQ under the kidney capsule with or without 1.0×10⁶ ILC2s or ILC3s and monitored for blood glucose levels. After 30 days, mice were transfused with 1.5×106 HLA-A2-specific CD4 and CD8 CAR-T cells with or without 1.0×106 ILC2s or ILC3s. On day 24 post-T cell infusion, mice were fasted for 4 hours and challenged with 1.5 g of glucose/kg (FIG. 10a). ILC2s and ILC3s prevent islet graft rejection in vivo. Following adoptive transfer of ILC2₁₀, rejection of islets is completely abrogated.

HLA-A2-specific CAR T cells or Naïve CD4+ T cells isolated using FACS sorting were plated with ILC2s or ILC3s (1:1 ratio) and stimulated with anti-CD3/CD28 Dyna beads for 2 or 4 days, respectively. Surface and intracellular markers were quantified by flow cytometry. Intracellular cytokine production was quantified by flow cytometry after a 6-hour stimulation with PMA/Ionocmycin (FIGS. 10b and 10c). ILC2s and ILC3s suppress HLA-A2-specific CAR CD4 and CD8 T cells by limiting IFN-γ secretion, FasL expression and proliferation. ILC2s limit pro-inflammatory IFN-γ from naïve CD4⁺ T cells in both an autologous and allogeneic context.

In vitro co-culture of ILC2₁₀ with HLA-A2+ CART cells demonstrates ILC2₁₀ directly inhibit T cell proliferation (Ki67) (FIG. 10d) and CD4+ and CD8+ T cell production of proinflammatory cytokines IFN-γ and TNF-α (FIG. 10e).

### Discussion

The results in this study highlight the potential for human ILC2s to suppress inflammatory T cell functions and limit aGVHD pathology. Previous studies in mice and humans have shown that ILCs, in particular ILC2s, are acutely sensitive to pre-transplant conditioning therapies and are depleted from both peripheral blood and tissues^{14,43,44}. After engraftment, helper ILCs are slow to reconstitute, unlike NK cells, further resulting in a reduction in helper-like ILCs after HSCT. Our results are in line with prior studies which demonstrated a high proportion of helper ILC reconstitution is correlated with patients that do not develop aGVHD⁴⁴.

Recently, it was shown that while the proportion of CD34⁺ cells in the HSCT graft did not correlate to ILC recovery in the HSCT recipient, the relative proportion of total CD127⁺ ILCs in the graft did correlate in a small cohort of patients^{43,44}. Patients who received grafts with relatively low proportions of ILCs had an increased risk of developing aGVHD compared to patients with high proportions of ILCs, as did patients with a relatively low proportion of ILCs post-induction but pre-HSCT⁴⁴. This supports the putative protective roles of ILCs in aGVHD within the donor graft. From a therapeutic perspective, this could mean delivery of in vitro expanded IL-10-producing ILC2s in the donor HSC graft itself or infused into the patient along with HSCT after conditioning therapy might have benefits in reducing GVHD.

Analysis of T cells in vitro and in vivo revealed that expanded IL-10-producing ILC2s directly modulated T cells through production of IL-4 and IL-10. These results are supportive of previous findings that mouse ILC2s were protective against intestinal GVHD, but here we report a distinct mechanism utilized by human ILC2s. Bruce et al. reported that transfer of mouse ILC2s ameliorated aGVHD in a major MHC-mismatch mouse model of GVHD¹⁴, but that this was driven by MDSC recruitment to the gastrointestinal tract and activation by ILC2-derived IL-13. Here, we report human ILC2s that produce IL-10 have direct ability to regulate T cell responses in a xenograft GVHD model. The vast majority of human CD45⁺ cells engrafted after PBMC injection are T cells, with limited contribution from myeloid and B cells. These results suggest ILC2s may have multiple mechanisms of protection from GVHD.

Recent studies have revealed the diverse contexts in which IL-10-producing ILC2s limit inflammation. Lung ILC2s from mice injected with IL-33 showed a dramatic induction in IL-10 expression which was enhanced with IL-216. This phenotype could be replicated in vitro by culturing ILC2s with IL-2 and retinoic acid¹⁶. ILCs from nasal tissue taken from individuals with grass-pollen allergies had reduced capacity to produce IL-10 following stimulation¹⁷. Patients who received grass-pollen immunotherapy had increased proportions of IL-10⁺ ILC2s and this correlated with reduced disease severity²⁰. Additionally, mouse ILC2s were found to be the primary producers of intestinal IL-10 during both steady state and disease and key regulators of intestinal inflammation¹⁹. In the context of transplantation, Huang et al. demonstrated murine IL-10-producing ILC2s could prolong islet allograft survival by limiting allogeneic T cell responses to the islets³⁴. These growing reports demonstrate central roles for IL-10-producing ILC2s in regulating immunity and taken with our study, support that adoptive transfer of ILC2₁₀ may have applications in tolerance promoting cell therapies.

Findings from multiple groups, along with ours, have consistently found that CD127⁺CRTh2⁺ ILC2s are a heterogeneous group of cells^{15,16,18-20,34}. While the bulk expanded ILC2s successfully limited the development of xenograft GVHD, this may be enhanced by isolating and expanding only the ILC2s that express IL-4 and IL-10. However, there has been limited research towards identifying markers that are exclusively expressed on IL-10⁺ ILC2s. In one of the few studies that considered this, Golebski et al. found that it was only the KLRG1⁺ ILC2s that had the ability to produce IL-10 along with other signature ILC2 cytokines²⁰. However, CITE-seq on IL-10⁺ ILC2s before and after expansion in our study revealed limited expression of KLRG1 relative to other ILC subsets, suggesting other markers may be needed to identify and isolate these cells from healthy blood donors.

Our results highlight that in vitro expanded human ILC2s are directly able to regulate allogeneic CD4⁺ and CD8⁺ T cell responses and suppress the development of xenograft GVHD. Similarly, recent research by Bruce et al. in mice revealed that 3^{rd} party ILC2s could improve the survival of mice in an MHC-mismatch model of aGVHD⁴⁵. In a cell therapy context, this would allow for the use of ILC2s not only from the HSCT donor or recipient, but also from 3^{rd} party donors, alleviating time constraint issues in acquiring, expanding, and functionally testing ILC2s from the HSCT donor or recipient. Future research on the viability and function of cryopreserved ILC2s would emphasize the potential of ILC2s as an ‘off-the-shelf’ treatment for aGVHD.

ILC2s can be isolated and expanded from healthy human blood and post-expansion, ILC2s maintain expression of signature cytokines. A single infusion of expanded allogeneic ILC2s into a xenograft GVHD mouse model was able to reduce the severity of xenograft GVHD symptoms and improve overall NSG mouse survival. CD4⁺ and CD8⁺ T cells had reduced expression of Ki-67 and CXCR3 in vivo after allogeneic ILC2 treatment, indicating reduced proliferation, trafficking the gastrointesintestinal tract and Th1 and Tc1 polarization. In vitro co-cultures revealed allogeneic ILC2s were able to reduce expression of IFN-γ in CD4⁺ and CD8⁺ T cells and this was ameliorated by blocking IL-4 and IL-10 together. Altogether, our findings support that human IL-10-producing ILC2s limit harmful allogeneic T cell responses and may have applications in adoptive cell-based therapies aimed at limiting harmful immune responses.

Although preferred embodiments of the invention have been described herein, it will be understood by those skilled in the art that variations may be made thereto without departing from the spirit of the invention or the scope of the appended claims. All documents disclosed herein, including those in the following reference list, are incorporated by reference.

### REFERENCE LIST

1. Garnett, C., Apperley, J. F. & Pavlu, J. Treatment and management of graft-versus-host disease: improving response and survival. Ther Adv Hematol 4, 366-78 (2013).
2. Xhaard, A. et al. Steroid-Refractory Acute GVHD: Lack of Long-Term Improved Survival Using New Generation Anticytokine Treatment. Biology of Blood and Marrow Transplantation 18, 406-413 (2012).
3. Cheung, T. S. et al. Mesenchymal Stromal Cells for Graft Versus Host Disease: Mechanism-Based Biomarkers. Frontiers in Immunology vol. 11 (2020).
4. Elias, S. & Rudensky, A. Y. Therapeutic use of regulatory T cells for graft-versus-host disease. British Journal of Haematology vol. 187 25-38 (2019).
5. Koehn, B. H. & Blazar, B. R. Role of myeloid-derived suppressor cells in allogeneic hematopoietic cell transplantation. J Leukoc Biol 102, 335-341 (2017).
6. Vivier, E. et al. Innate Lymphoid Cells: 10 Years On. Cell vol. 174 1054-1066 (2018).
7. Spits, H. et al. Innate lymphoid cells—a proposal for uniform nomenclature. Nature Reviews Immunology 13, 145-149 (2013).
8. de Maria, A., Bozzano, F., Cantoni, C. & Moretta, L. Revisiting human natural killer cell subset function revealed cytolytic CD56dimCD16+ NK cells as rapid producers of abundant IFN-γ on activation. Proc Natl Acad Sci USA 108, 728-732 (2011).
9 Simonetta, F., Alvarez, M. & Negrin, R. S. Natural killer cells in graft-versus-host-disease after allogeneic hematopoietic cell transplantation. Frontiers in Immunology vol. 8 (2017).
10. Minculescu, L. et al. Early Natural Killer Cell Reconstitution Predicts Overall Survival in T Cell-Replete Allogeneic Hematopoietic Stem Cell Transplantation. Biology of Blood and Marrow Transplantation 22, 2187-2193 (2016).
11. Marius Munneke, J. et al. Activated innate lymphoid cells are associated with a reduced susceptibility to graft-versus-host disease. Blood 124, 812-821 (2014).
12. Hanash, A. M. et al. Interleukin-22 Protects Intestinal Stem Cells from Immune-Mediated Tissue Damage and Regulates Sensitivity to Graft versus Host Disease. Immunity 37, 339-350 (2012).
13. Hazenberg, M. D. et al. Human ectoenzyme-expressing ILC3: Immunosuppressive innate cells that are depleted in graft-versus-host disease. Blood Advances 3, 3650-3660 (2019).
14. Bruce, D. W. et al. Type 2 innate lymphoid cells treat and prevent acute gastrointestinal graft-versus-host disease. Journal of Clinical Investigation 127, 1813-1825 (2017).
15. Jegatheeswaran, S., Mathews, J. A. & Crome, S. Q. Searching for the Elusive Regulatory Innate Lymphoid Cell. The Journal of Immunology 207, 1949-1957 (2021).
16. Seehus, C. R. et al. Alternative activation generates IL-10 producing type 2 innate lymphoid cells. Nature Communications 8, (2017).
17. Howard, E. et al. IL-10 production by ILC2s requires Blimp-1 and cMaf, modulates cellular metabolism, and ameliorates airway hyperreactivity. Journal of Allergy and Clinical Immunology 147, 1281-1295.e5 (2021).
18. Nc, D. et al. Meningeal type-2 innate lymphoid cells emerge as novel regulators of microglial activation and blood-brain barrier stability: a central role for IL-10. https://ssrn.com/abstract=3414004 (2019).
19. Bando, J. K. et al. ILC2s are the predominant source of intestinal ILC-derived IL-10. Journal of Experimental Medicine 217, (2020).
20. Golebski, K. et al. Induction of IL-10-producing type 2 innate lymphoid cells by allergen immunotherapy is associated with clinical response. Immunity 54, 291-307.e7 (2021).
21. Garnett, C., Apperley, J. F. & Pavlu, J. Treatment and management of graft-versus-host disease: Improving response and survival. Therapeutic Advances in Hematology vol. 4 366-378 (2013).
22. Crome, S. Q., Wang, A. Y., Kang, C. Y. & Levings, M. K. The role of retinoic acid-related orphan receptor variant 2 and IL-17 in the development and function of human CD4+ T cells. European Journal of Immunology 39, 1480-1493 (2009).
23. Crome, S. Q. et al. Inflammatory Effects of Ex Vivo Human Th17 Cells Are Suppressed by Regulatory T Cells. The Journal of Immunology 185, 3199-3208 (2010).
24. Acosta-Rodriguez, E. v. et al. Surface phenotype and antigenic specificity of human interleukin 17-producing T helper memory cells. Nature Immunology 8, 639-646 (2007).
25. Guy-Grand, D. & Vassalli, P. Gut injury in mouse graft-versus-host reaction. Study of its occurrence and mechanisms. Journal of Clinical Investigation 77, 1584-1595 (1986).
26. Szebeni, J., Wang, M. G., Pearson, D. A., Szot, G. L. & Sykes, M. IL-2 inhibits early increases in serum gamma interferon levels associated with graft-versus-host-disease. Transplantation 58, 1385-93 (1994).
27. Mortha, A. & Burrows, K. Cytokine networks between innate lymphoid cells and myeloid cells. Frontiers in Immunology vol. 9 (2018).
28. Ehx, G. et al. Xenogeneic Graft-Versus-Host Disease in Humanized NSG and NSG-HLA-A2/HHD Mice. Frontiers in Immunology 9, (2018).
29. Croudace, J. E. et al. Chemokine-mediated tissue recruitment of CXCR3 CD4 T cells plays a major role in the pathogenesis of chronic GVHD. (2012) doi: 10.1182/blood.
30. Choi, J. et al. IFNR signaling mediates alloreactive T-cell trafficking and GVHD. http://ashpublications.org/blood/article-pdf/120/19/4093/1360293/zh804512004093.pdf (2012).
31. Burman, A. C. et al. IFNγ differentially controls the development of idiopathic pneumonia syndrome and GVHD of the gastrointestinal tract. Blood 110, 1064-1072 (2007).
32. Gutierrez-Hoya, A. et al. Role of CD8 Regulatory T Cells versus Tc1 and Tc17 Cells in the Development of Human Graft-versus-Host Disease. Journal of Immunology Research 2017, (2017).
33. Cosmi, L. et al. CRTH2 is the most reliable marker for the detection of circulating human type 2 Th and type 2 T cytotoxic cells in health and disease.
34. Huang, Q. et al. IL-10 producing type 2 innate lymphoid cells prolong islet allograft survival. EMBO Molecular Medicine 12, (2020).
35. Chen, P. P. et al. Alloantigen-specific type 1 regulatory T cells suppress through CTLA-4 and PD-1 pathways and persist long-term in patients. Science Translational Medicine 13, (2021).
36. Gagliani, N. et al. Coexpression of CD49b and LAG-3 identifies human and mouse T regulatory type 1 cells. Nature Medicine 19, 739-746 (2013).
37. Long, A. et al. Type 2 Innate Lymphoid Cells Impede IL-33-Mediated Tumor Suppression. The Journal of Immunology 201, 3456-3464 (2018).
38. Ohta, A. et al. A2A Adenosine Receptor May Allow Expansion of T Cells Lacking Effector Functions in Extracellular Adenosine-Rich Microenvironments. The Journal of Immunology 183, 5487-5493 (2009).
39. Allard, B., Longhi, M. S., Robson, S. C. & Stagg, J. The ectonucleotidases CD39 and CD73: Novel checkpoint inhibitor targets. Immunological Reviews vol. 276 121-144 (2017).
40. Yang, L. et al. Interleukin-13 interferes with activation-induced t-cell apoptosis by repressing p53 expression. 13, (2015).
41. Rauber, S. et al. Resolution of inflammation by interleukin-9-producing type 2 innate lymphoid cells. Nature Medicine 23, 938-944 (2017).
42. Murphy, K. M. & Reiner, S. L. The lineage decisions of helper T cells. Nature Reviews Immunology vol. 2 933-944 (2002).
43. Piperoglou, C. et al. Innate lymphoid cell recovery and occurrence of GvHD after hematopoietic stem cell transplantation. Journal of Leukocyte Biology 111, 161-172 (2022).
44. Kroeze, A. et al. Presence of innate lymphoid cells in allogeneic hematopoietic grafts correlates with reduced graft-versus-host disease. Cytotherapy (2022) doi: 10.1016/j.jcyt.2021.10.011.
45. Bruce, D. W. et al. Third-party type 2 innate lymphoid cells prevent and treat GI tract GvHD. Blood Advances 5, 4578-4589 (2021).
46. Hao, Y. et al. Integrated analysis of multimodal single-cell data. Cell 184, 3573-3587.e29 (2021).
47. McInnes, L., Healy, J., Saul, N. & Großberger, L. UMAP: Uniform Manifold Approximation and Projection. Journal of Open Source Software 3, 861 (2018).

1. A method of ameliorating, treating or preventing graft-versus-host disease, transplant rejection or an autoimmune disorder, or promoting transplant graft function in a human subject in need thereof, the method comprising administering to the subject a therapeutically effective amount of a population of cells comprising innate lymphoid cells (ILCs).

2. The method of claim 1, wherein the population comprises ILC2 cells, ILC3 cells or ILCregs cells or combinations thereof.

3. The method of claim 2, wherein the population comprises ILC2 cells that produce IL-4, IL-5, IL-9 or IL-13.

4. The method of claim 1, wherein the ILC2 cells produce IL-10.

5. The method of claim 4, wherein the ILC2, cells additionally produce Amphiregulin.

6. The method of claim 4, wherein the ILC2 cells are CD45+, CD127+, CRTh2+ and CD3−, CD4−, CD8−, CD14−, CD15−, CD19−, CD20−, CD33−, CD34−, CD68−, CD138−, CD203c−, FCεRI−, TCRαβ−, TCRγδ−, IgA−, CD16−, CD94−, NKG2D−, CCR6− and NKp44−.

7. The method of claim 2, wherein the population comprises ILC3 cells that produce IL-22 alone or in combination with IL-17 or GM-CSF.

8. The method of claim 7, wherein the ILC3 cells additionally produce IL-10 and Amphiregulin.

9. The method of claim 7, wherein the ILC3 cells are CD45+, CD127+, CD117+, CCR6+, CRTh2−, CD3−, CD4−, CD8−, CD14−, CD15−, CD19−, CD20−, CD33−, CD34−, CD68−, CD138−, CD203c−, FCεRI−, TCRαβ−, TCRγδ−, IgA−, CD16−, CD94− and NKG2D−.

10. The method of claim 2, wherein the population comprises ILCregs that are CD7+, NKp44+, NKp46+, express Galectin-9 and HELIOS, do not express IL-10, and have low to no expression of IFN-γ and TNF-α.

11. The method of claim 2, wherein the ILCs are CD56+CD3−.

12. The method of claim 11, wherein the ILCregs are further at least one of CD56hi, CD16−, CD7+, CD94+, NKG2D+, KIR+, GITR, NKp44− ex vivo, NKp46+, IL-22+, do not express IL-10 and have low to no expression of IFN-γ and TNF-α.

13. The method of claim 12, wherein the ILCregs are further all of CD56hi, CD16−, CD7+, CD94+, NKG2D+, KIR+, GITR, NKp44− ex vivo, NKp30+, NKp46+, IL-22+, do not express IL-10 and have low to no expression of IFN-γ and TNF-α.

14. The method of claim 1, wherein the ILCs suppress T-cell function or propagation.

15. The method of claim 1, wherein the subject has undergone or will undergo a cell transplantation procedure.

16. The method of claim 15, wherein the cell transplantation is stem cell transplantation, the latter preferably being hematopoietic stem cell transplantation.

17. The method of claim 1, wherein the subject has undergone or will undergo a bone marrow transplantation procedure.

18. The method of claim 1, wherein the subject has undergone or will undergo a solid organ transplantation procedure.

19. (canceled)

20. (canceled)

21. A method for inducing a population of regulatory innate lymphoid cells, (ILCregs) from innate lymphoid cell (ILC) precursors and/or NK cells, and the method comprising culturing the population with at least one of TGF-β, IL-2 and anti-IFN-γ.

22-30. (canceled)

31. A method of selecting from a population of cells comprising innate lymphoid cells (ILCs), those cells that are ILC2 cells by selecting for cells that are CD45+, CD127+, CRTh2+ and CD3−, CD4−, CD8−, CD14−, CD15−, CD19−, CD20−, CD33−, CD34−, CD68−, CD138−, CD203c−, FCεRI−, TCRαβ−, TCRγδ—, IgA−, CD16−,CD94−, NKG2D−, CCR6− and NKp44−.

32-46. (canceled)
